(19) United States
(12) Patent Application Publication
(10) Pub. No.: US 2013/0295016 A1
(43) Pub. Date: Nov. 7, 2013

(54) SIGNATURES OF ELECTROENCEPHALOGRAPHIC OSCILLATIONS

(71) Applicants: David J. Gerber, Somerville, MA (US); Margaret E. Levin, Cambridge, MA (US); Jonathan M. Levenson, Walpole, MA (US); Kevin M. Spencer, Quincy, MA (US)

(72) Inventors: David J. Gerber, Somerville, MA (US); Margaret E. Levin, Cambridge, MA (US); Jonathan M. Levenson, Walpole, MA (US); Kevin M. Spencer, Quincy, MA (US)

(21) Appl. No.: 13/889,265

(22) Filed: May 7, 2013

Related U.S. Application Data
(60) Provisional application No. 61/643,581, filed on May 7, 2012.

(51) Int. Cl. A61B 5/16

(52) U.S. Cl. CPC A61B 5/16
USPC 424/9.2; 600/544

(57) ABSTRACT

The present invention provides, in some aspects, methods for identifying and evaluating signatures in electroencephalographic oscillations that occur during onset of an exploratory activity in a subject.

### RELATED APPLICATIONS

This application claims priority under 35 U.S.C. §119(e) to U.S. Provisional Application Ser. No. 61/643,581, entitled “SIGNATURES OF ELECTROENCEPHALOGRAPHIC OSCILLATIONS” filed on May 7, 2012, which is herein incorporated by reference in its entirety.

### FEDERALLY SPONSORED RESEARCH

This invention was created in the performance of a Cooperative Research and Development Agreement with the Department of Veterans Affairs, an agency of the U.S. Government, which has certain rights in this invention

### FIELD OF INVENTION

The present invention relates to methods for identifying and evaluating neurological events. In some aspects, the invention provides methods for detecting deficiencies in neurological events in a subject and for identifying agents that modulate neurological events in a subject.

### BACKGROUND OF THE INVENTION

Discovery of new commercially viable therapeutics for central nervous system (CNS) disorders has significantly lagged behind other therapeutic areas, with some estimates suggesting only a 1% success rate for new chemical entities in the United States. Several factors have contributed to the difficulty in discovering effective, new CNS therapeutics including, for example, a lack of disease-relevant functional screens, a lack of clinically predictive animal models and an absence of reliable and specific biomarkers for use as diagnostics and objective measures of drug efficacy. These challenges have particularly impacted the discovery of cognitive therapies for schizophrenia, and other disorders. For example, there are currently no effective drugs for treatment of the cognitive or negative symptoms of the spectrum of diseases related to schizophrenia. Disease-relevant biomarkers that are useful for translating basic preclinical discoveries into effective therapeutics can facilitate the development of therapeutics for CNS disorders.

### SUMMARY OF INVENTION

Aspects of the invention are based on the recognition that the development of new and effective therapeutics for cognitive disorders has been challenged by the complexity and multi-factorial nature of cognitive disorders and the lack of objective functional measures of the neural circuitry whose alteration underlies the cognitive deficits associated with such disorders. According to some aspects of the invention, approaches are provided that identify specific features of neural activity that are altered in cognitive disorders. According to some aspects of the invention, disease-relevant biomarkers are provided that serve as a basis for translating basic preclinical discoveries into effective therapeutics. In some embodiments, biomarkers based on electrophysiological endophenotypes serve as objective indicators of cognitive disease states. In some embodiments, biomarkers are provided that are useful for evaluating drug candidate efficacy during clinical trials and for developing personalized treatment regimes. Some aspects of the invention are based on the discovery that specific alterations in neural activity occur in a subject during onset of an exploratory activity. In some embodiments, these specific alterations in neural activity are believed to influence attention, cognition, memory, and/or learning in connection with the exploratory activity. In some embodiments, these specific alterations in neural activity during onset of an exploratory activity are associated with dopamine receptor activity.

Certain aspects of the invention relate to the discovery of certain signatures in electroencephalographc oscillations recorded from normal subjects. In some embodiments, these signatures are absent in subjects having schizophrenia and related disorders associated with cognitive deficits. In some embodiments, the signatures serve as biomarkers for one or more cognitive deficits. In certain embodiments, the electroencephalographic (EEG) oscillations provide a basis for diagnosing or monitoring a cognitive deficit in a subject based on changes in the EEG oscillations during onset of an exploratory activity. In some embodiments, the EEG oscillations provide a basis for identifying candidate therapeutic agents for treating cognitive deficits based on changes in the EEG oscillations during onset of an exploratory activity. In some embodiments, the EEG oscillations provide a basis for monitoring the effectiveness of therapeutic agents for treating cognitive disease.

In some aspects of the invention, methods are provided that involve determining the presence or absence of a signature in electroencephalographic oscillations recorded from a subject during onset of an exploratory activity engaged in by the subject. In some embodiments, presence of the signature in the electroencephalographic oscillations is indicative of absence of a cognitive disorder in the subject, and absence of the signature in the electroencephalographic oscillations is indicative of presence of the cognitive disorder in the subject.

In some aspects of the invention, methods are provided that involve administering a test agent to subject who is identified as having a cognitive disorder; and determining the presence or absence of a signature in electroencephalographic oscillations recorded from the subject during onset of an exploratory activity engaged in by the subject after having been administered the test agent. In some embodiments, presence of the signature in the electroencephalographic oscillations is indicative of effectiveness of the test agent in treating the cognitive disorder, and absence of the signature in the electroencephalographic oscillations is indicative of a lack of effectiveness of the test agent in treating the cognitive disorder.

In some aspects of the invention, methods are provided for diagnosing, or aiding in diagnosing, a subject as having a cognitive disorder. In some embodiments, the methods involve identifying a subject suspected of having a cognitive disorder or at risk of having the cognitive disorder; and determining the presence or absence of a signature in electroencephalographic oscillations recorded from the subject during onset of an exploratory activity engaged in by the subject. In some embodiments, presence of the signature in the electroencephalographic oscillations is indicative of absence of a cognitive disorder in the subject, and absence of the signature in the electroencephalographic oscillations is indicative of presence of the cognitive disorder in the subject.

In some embodiments, the methods disclosed herein involve recording electroencephalographic oscillations from the subject during onset of the exploratory activity. In some embodiments, the methods involve stimulating the subject to engage in the exploratory activity.

In some embodiments, the signature is based on power of the electroencephalographic oscillations or a phase-locking characteristic of the electroencephalographic oscillations. In some embodiments, the signature is a first maxima of power of the electroencephalographic oscillations occurring within a first frequency band followed by a second maxima of power of the electroencephalographic oscillations occurring within a second frequency band. In certain embodiments, the second maxima occurs 10 milliseconds to 1000 milliseconds following the first maxima. In certain embodiments, the first frequency band comprises lower frequencies than that second frequency band. In certain embodiments, the first frequency band is in a range of 10 Hz to 30 Hz. In certain embodiments, the second frequency band is in a range of 60 Hz to 100 Hz.

In some embodiments, the exploratory activity is engaged in by the subject when an appropriate stimulus is in the perceptual environment of the subject. In some embodiments, the methods further involve setting an appropriate stimulus in the perceptual environment of the subject. In some embodiments, the appropriate stimulus is an object or image. In some embodiments, the appropriate stimulus comprises a light, sound, odorant, tastant, or tactile stimulant. In some embodiments, the appropriate stimulus induces the subject's sense of sight, hearing, smell, taste or touch. In some embodiments, prior to the appropriate stimulus being set in the perceptual environment, the subject has not been exposed to the appropriate stimulus for at least 12 hours, at least 24 hours or at least 48 hours. In some embodiments, prior to the appropriate stimulus being set in the perceptual environment, the subject has not been exposed to the appropriate stimulus. In some embodiments, the exploratory activity involves a body portion of the subject being maintained within a first distance from the object for a first period. In some embodiments, initiation of the exploratory activity occurs when the body portion of the subject enters within the first distance. In some embodiments, the body portion is the subject's mid-torso, limb, finger, hand, foot, nose, paw, snout, or vibrissae. In some embodiments, initiation of the exploratory activity occurs when the image is presented in the perceptual environment of the subject.

In some embodiments, the presence or absence of the signature is determined in electroencephalographic oscillations recorded from 3 seconds prior to initiation of the exploratory activity to 3 seconds after initiation of the exploratory activity. In some embodiments, the presence or absence of the signature is determined in electroencephalographic oscillations recorded from 3 second prior to initiation of the exploratory activity to 1 second after initiation of the exploratory activity. In some embodiments, the presence or absence of the signature is determined in electroencephalographic oscillations recorded from 2 seconds prior to initiation of the exploratory activity to initiation of the exploratory activity. In some embodiments, the presence or absence of the signature is determined in electroencephalographic oscillations recorded from 1 second prior to initiation of the exploratory activity to 3 seconds after initiation of the exploratory activity. In some embodiments, the presence or absence of the signature is determined in electroencephalographic oscillations recorded from initiation of the exploratory activity to 2 seconds after initiation of the exploratory activity.

In some embodiments, the subject is a mammal. In some embodiments, the subject is a rodent. In certain embodiments, the rodent is a mouse or a rat. In some embodiments, the subject is a primate. In certain embodiments, the primate is a non-human primate. In certain embodiments, the primate is a human.

In some embodiments, the cognitive disorder is associated with a calcineurin deficiency. In some embodiments, the cognitive disorder is schizophrenia, bipolar disorder, Alzheimer's disease, Parkinson's disease, Huntington's disease, multiple sclerosis, Attention Deficit Hyperactivity Disorder (ADHD), autism, a learning disorder, a memory disorder, an injury, or anxiety. In some embodiments, the cognitive disorder is a chemically induced cognitive disorder. In certain embodiments, the chemically induced cognitive disorder is induced with a compound that impairs glutamatergic function, a compound that enhances dopaminergic function, a compound that modulates serotonin function, a hallucinogenic compound, or a compound that impairs cholinergic function. In certain embodiments, the chemically induced cognitive disorder is induced with phencyclidine (PCP), MK-801, 3-(2-Carboxypiperazin-4-yl)propyl-1-phosphonic acid (CPP), ketamine, apomorphine, D-amphetamine, methamphetamine, mescaline, lysergic acid diethylamide (LSD), an opioid, a cannabinoid, psilocybin, scopolamine, or atropine. In some embodiments, the cognitive disorder is associated with a genetic alteration. In certain embodiments, the genetic alternation disrupts calcineurin signalling. In certain embodiments, the subject is a calcineurin knock-out mouse (CNKO mouse). In certain embodiments, calcineurin is knocked out postnatally in forebrain neurons of the mouse.

In some embodiments, the recorded electroencephalographic oscillations emanate from at least the prefrontal cortex, the striatum or the hippocampus of the subject. In some embodiments, the recorded electroencephalographic oscillations emanate from at least the prefrontal cortex of the subject. In certain embodiments, the recorded electroencephalographic oscillations emanate from at least a midbrain dopaminergic region of the subject. In certain embodiments, the midbrain dopaminergic region is a ventral tegmental region. In some embodiments, the recorded electroencephalographic oscillations emanate from at least a brain region comprising the frontal association cortex.

In some embodiments, the electroencephalographic oscillations are recorded from an implanted electrode. In certain embodiments, the implanted electrode is a subdural or epidural electrode. In some embodiments, the electroencephalographic oscillations are recorded from an external electrode. In certain embodiments, the external electrode is a scalp electrode or an electrode cap.

In some embodiments, the subject is a mouse and the electroencephalographic oscillations are recorded from a region of brain that is within medial-lateral extent posterior to the olfactory bulb, anterior to M2 motor cortex, and superficial to orbital cortex. In certain embodiments, the subject is a mouse and recording electroencephalographic oscillations comprises recording from a brain area having the coordinates: from Bregma +0.37 mm rostral, +0.07 mm lateral, −0.05 mm deep from the brain surface.

### BRIEF DESCRIPTION OF DRAWINGS

FIG. 1A depicts a process for analyzing signatures of EEG oscillations;

FIG. 1B depicts a time-frequency map derived from EEG oscillations;

FIG. 1C depicts time-frequency map derived from EEG oscillations and projections into frequency and time domains;

FIG. 2 provides a schematic of a novel object recognition task configuration;

FIG. 3 provides examples of objects for a novel object recognition task;

FIG. 4 provides an image of a rodent engaged in an exploratory task during a novel object recognition task;

FIG. 5A. provides bar graphs that quantify percentage time engaged in an exploratory activity associated with exposure to a familiar object and exposure to a novel object for control and CNₕₑₜKO mice;

FIG. 5B depicts representative Gamma_{Hi} (65-90 Hz) bandpass filtered electroencephalographic (EEG) traces spanning from a time before to a time during an exploratory activity for control and CNₕₑₜKO mice;

FIG. 5C depicts bar graphs quantifying power in Gamma_{Hi} (65-90 Hz) frequency band;

FIG. 5D depicts bar graphs quantifying power in Theta (4-12 Hz) frequency band;

FIG. 6 depicts bar graphs quantifying power in Gamma_{Hi} (65-90 Hz) frequency band in untreated CNₕₑₜKO mice and in CNₕₑₜKO mice treated with PD 168077 (at different times following the treatment);

FIG. 7A depicts time-frequency maps derived from EEG oscillations for wild-type and CNₕₑₜKO mice;

FIG. 7B depicts bar charts showing time spent exploring novel and familiar objects for wild-type and CNₕₑₜKO mice;

FIG. 7C depicts time-frequency maps derived from EEG oscillations for subjects grouped based on exploratory activity performance;

FIG. 8A depicts time-frequency maps derived from EEG oscillations for CNₕₑₜKO mice with and without treatment with D4 Agonist PD168077;

FIG. 8B depicts bar graphs quantifying percentage time engaged in an exploratory activity associated with exposure to a familiar object and exposure to a novel object in CNₕₑₜKO mice treated with cyclodextrin or PD 168077;

FIG. 9A depicts time-frequency maps derived from EEG oscillations for wild-type mice and Coloboma mice;

FIG. 9B depicts bar graphs quantifying percentage time engaged in an exploratory activity associated with exposure to a familiar object and exposure to a novel object for wild-type mice and Coloboma mice;

FIG. 10 depicts time-frequency maps derived from EEG oscillations for untreated wild-type mice and wild-type mice treated with PCP;

FIG. 11 depicts examples of images used as visual stimuli for a novelty oddball paradigm;

FIG. 12A depicts a statistical time-frequency map showing clusters of thresholded p-values indicating the significant of differences in phase-locking factors between novel and dim images in a novelty oddball paradigm;

FIG. 12B depicts statistical interaction plots between healthy controls and schizophrenic subjects for each of the three clusters of thresholded p-values in FIG. 12A;

FIG. 13A depicts a statistical time-frequency map showing clusters of thresholded p-values indicating the significant of differences in phase-locking factors between novel and dim images in a novelty oddball paradigm; and

FIG. 13B depicts statistical interaction plots between healthy controls and schizophrenic subjects for each of the five clusters of thresholded p-values in FIG. 13A.

### DETAILED DESCRIPTION OF INVENTION

According to some aspects of the invention, specific alterations in neural activity occur in a subject during onset of an exploratory activity engaged in by the subject. The specific alterations may be detected by analyzing EEG oscillations obtained from a subject during onset of the exploratory activity. The EEG oscillations may be analyzed to determine, for example, the power of EEG oscillations during onset of the exploratory activity. In certain embodiments, specific alterations in the power of EEG oscillations during onset of the exploratory activity are alterations that are indicative of a cognitive disorder. In other embodiments, the EEG oscillations may be analyzed to determine, for example, phase locking characteristics of EEG oscillations during onset of the exploratory activity. In certain embodiments, specific alterations in phase locking characteristics of EEG oscillations during onset of the exploratory activity are alterations that are indicative of a cognitive disorder.

Certain aspects of the invention, relate to the discovery of deficiencies in neural activity during onset of an exploratory activity that are observed in subjects who have schizophrenia or related disorders associated with cognitive deficits. Accordingly, it has been discovered that subjects having certain diseases associated with cognitive deficits exhibit characteristic alterations in neural activity during onset of an exploratory activity. In certain embodiments, the electroencephalographic oscillations provide a basis for diagnosing or monitoring a cognitive disorder in a subject based on changes in the EEG oscillations during onset of an exploratory activity. In some embodiments, the EEG oscillations provide a basis for identifying candidate therapeutic agents for treating cognitive disorders based on changes in the EEG oscillations during onset of an exploratory activity. These and other embodiments of the invention are described in greater detail below.

### Neural Activity During Onset of an Exploratory Activity

Methods are provided herein for assessing neural activity that occurs during onset of an exploratory activity in a subject. As used herein, the term “exploratory activity” refers to an activity engaged in by a subject that employs the subject's investigative, probing, attending, or examining faculties. An exploratory activity typically occurs under controlled experimental conditions and over a period of time having a distinct point of initiation. An exploratory activity may be brought about by exposing a subject to an appropriate stimulus that invokes one or more of the subject's senses (e.g., smell, sight, hearing, taste, direction, acceleration, balance, etc.). As used herein, the term “sense” refers to a function or mechanism by which a subject receives and/or processes external or internal stimuli or by which a subject discerns its direction, position, speed and/or acceleration relative to its surroundings. The stimulus that invokes the subject's sense(s) may be, for example, an object, an image, an odor, a light source, a sound or a combination thereof.

In some embodiments, an exploratory activity is an activity that invokes an unconditioned response in a subject. In such embodiments, an exploratory activity invokes an unlearned, innate, spontaneous or involuntary response in a subject. An exploratory activity, in such embodiments, is not an activity for which a subject has been previously instructed, trained or conditioned to respond in a particular manner (e.g., to a sensory cue). Accordingly, in such embodiments, an exploratory activity is not an activity for which a subject has been instructed, trained or conditioned to associate a particular sensory cue (e.g., smell, taste, sound, sight, etc.) with (a.) an unpleasant response (e.g., nausea, vomiting, unpleasant noise, electrical shock, heat, drowning sensation, etc.) that results or will result in avoidance of that particular sensory cue by the subject and/or (b.) a pleasant response (e.g., a reward, such as a food reward, a water reward, an emotional reward, a monetary reward, etc.) that results or will result in pursuit of that particular sensory cue by the subject.

An exploratory activity, in some embodiments, is not an activity for which a subject is instructed or trained to respond to a sensory cue within a particular period of time (e.g., as fast as possible) by, for example, performing or refraining from a particular task (e.g., pushing a button, pulling a lever, making a sound, observing a visual cue, etc.). In other embodiments, an exploratory activity is an activity for which a subject is instructed or trained to respond to a sensory cue within a particular period of time (e.g., as fast as possible) by, for example, performing or refraining from a particular task (e.g., pushing a button, pulling a lever, making a sound, observing a visual cue, etc.).

In some embodiments, the methods involve inducing a subject to initiate an exploratory activity and recording electroencephalographic oscillations from the subject during onset of an exploratory activity. The electroencephalographic oscillations may be recorded over a continuous recording session encompassing onset of the exploratory activity. Electroencephalographic oscillations may be recorded before the onset of the exploratory activity, during onset of the exploratory activity, during the exploratory activity and/or after the exploratory activity.

As used herein, the phrase, “onset of an exploratory activity,” refers to a predetermined period encompassing a point in time when initiation of an exploratory activity occurs. In some embodiments, initiation of an exploratory activity occurs when an appropriate stimulus is set within the perceptual environment of a subject. In some embodiments, initiation of an exploratory activity occurs when a body portion (e.g., head, torso, hand, etc.) of a subject enters within a particular distance from an appropriate stimulus (e.g., an object).

Onset of an exploratory activity may span from 10 sec prior to initiation of an exploratory activity to 10 sec after initiation. Onset of an exploratory activity may span from 5 sec prior to initiation of an exploratory activity to 5 sec after initiation. Onset of an exploratory activity may span from 3 sec prior to initiation of an exploratory activity to 3 sec after initiation. Onset of an exploratory activity may span from 2 sec prior to initiation of an exploratory activity to 2 sec after initiation. Onset of an exploratory activity may span from 1 sec prior to initiation of an exploratory activity to 1 sec after initiation.

Onset of an exploratory activity may span from 5 sec prior to initiation of an exploratory activity to 1 sec after initiation. Onset of an exploratory activity may span from 4 sec prior to initiation of an exploratory activity to 1 sec after initiation. Onset of an exploratory activity may span from 3 sec prior to initiation of an exploratory activity to 1 sec after initiation. Onset of an exploratory activity may span from 2 sec prior to initiation of an exploratory activity to 1 sec after initiation.

Onset of an exploratory activity may span from 5 sec prior to initiation of an exploratory activity to the point of initiation. Onset of an exploratory activity may span from 4 sec prior to initiation of an exploratory activity to the point of initiation. Onset of an exploratory activity may span from 3 sec prior to initiation of an exploratory activity to the point of initiation. Onset of an exploratory activity may span from 2 sec prior to initiation of an exploratory activity to the point of initiation. Onset of an exploratory activity may span from 1 sec prior to initiation of an exploratory activity to the point of initiation.

Onset of an exploratory activity may span from 1 sec prior to initiation of an exploratory activity to 5 sec after initiation. Onset of an exploratory activity may span from 1 sec prior to initiation of an exploratory activity to 4 sec after initiation. Onset of an exploratory activity may span from 1 sec prior to initiation of an exploratory activity to 3 sec after initiation. Onset of an exploratory activity may span from 1 sec prior to initiation of an exploratory activity to 2 sec after initiation.

Onset of an exploratory activity may span from the point of initiation of an exploratory activity to 5 sec after initiation. Onset of an exploratory activity may span from the point of initiation of an exploratory activity to 4 sec after initiation. Onset of an exploratory activity may span from the point of initiation of an exploratory activity to 3 sec after initiation. Onset of an exploratory activity may span from the point of initiation of an exploratory activity to 2 sec after initiation. Onset of an exploratory activity may span from the point of initiation of an exploratory activity to 1 sec after initiation.

Onset of an exploratory activity may span from 1 sec after initiation of an exploratory activity to 5 sec after initiation. Onset of an exploratory activity may span from 200 msec after initiation of an exploratory activity to 5 sec after initiation. Onset of an exploratory activity may span from 200 msec after initiation of an exploratory activity to 4 sec after initiation. Onset of an exploratory activity may span from 200 msec after initiation of an exploratory activity to 3 sec after initiation. Onset of an exploratory activity may span from 200 msec after initiation of an exploratory activity to 2 sec after initiation. Onset of an exploratory activity may span from 200 msec after initiation of an exploratory activity to 1 sec after initiation.

### Exploratory Activity

A variety of methods are known in the art for engaging a subject in an exploratory activity. Non-limiting examples of such art-known methods are disclosed in K. Rutten et al., Automated Scoring of Novel Object Recognition in Rats, Journal of Neuroscience Methods 171 (2008) 72-77; J. M. Silvers, et al., Automation of the novel object recognition task for use in adolescent rats, Journal of Neuroscience Methods 166 (2007) 99-103; A. Ennaceur et al., A new one-trial test for neurobiological studies of memory in rats. 1: Behavioral data, Behavioral Brain Research 31 (1988) 47-59; R. S. Hammond et al., On the delay-dependent involvement of the hippocampus in object recognition memory, Neurobiology of Learning and Memory 82 (2004) 26-34; L. Malkova et al., One-Trial Memory for Object-Place Associations after Separate Lesions of Hippocampus and Posterior Parahippocampal Region in the Monkey, Journal of Neuroscience 23(5) (2003) 1956-1965; D. Bovet et al., Judgment of conceptual identity in monkeys, Psychonomic Bulletin & Review 8(3) (2001) 470-475; K. R. Daffner et al., The central role of the prefrontal cortex in directing attention to novel events, Brain 123 (2000) 927-939; H. Mahut et al., Hippocampal Resections Impair Associative Learning and Recognition Memory in the Monkey, Journal of Neuroscience 2(9) (1982) 1214-1229; J. L. Voss et al., Finding meaning in novel geometric shapes influences electrophysiological correlates of repetition and dissociates perceptual and conceptual priming, NeuroImage 49 (2010) 2879-2889; J. W. Young et al., Using the MATRICS to guide development of a preclinical cognitive test battery for research in schizophrenia, Pharmacology & Therapeutics 122 (2009) 150-202; and Courchesne E, Hillyard S A, Galambos R (1975). Stimulus novelty, task relevance, and the visual evoked potential in man. Electroencephalogr Clin Neurophysiol 39:131-143. The contents of each of the foregoing references, which relate to exploratory activity, are incorporated herein by reference in their entirety.

In some embodiments, methods involve exposing the subject to a stimulus that induces an exploratory activity. Any appropriate stimulus may be used to induce an exploratory activity, including, for example, a light, a sound, an odorant, a tastant, or a tactile stimulant. The stimulus is often of a type that engages one or more senses of the subject, such as for example, the subject's sense of sight, hearing, smell, taste or touch. The stimulus may be for example an object, e.g., an object having a particular appearance, e.g., texture, color, shape, etc., that stimulates a subject to engage in an exploratory activity that involves probing, investigating or examining the object. In some embodiments, exposing the subject to the stimulus involves locating the object in the perceptual environment of the subject. In some cases, exposing the subject to the object involves presenting an image of the object in the perceptual environment of the subject. Depending on the method used, the subject may or may not have been exposed to the stimulus prior to initiating the exploratory activity.

An object may be familiar to a subject or may be novel to a subject. An object that is familiar to a subject is referred to as a “familiar object” relative to the subject. A familiar object is typically an object (1) that the subject has previously been exposed to, (2) that, in the previous exposure, invoked the subject's investigative, probing, attending or examining faculties, and (3) that does not at a present time invoke the subject's investigative, probing, attending or examining faculties. In some cases, a familiar object need not be the actual object that a subject was previously exposed to, but rather may be an object of the same type (e.g., an object of the same size, color, texture, shape, etc.) as the object that a subject was previously exposed to.

An object that is novel to a subject is referred to as a “novel object” relative to the subject. A novel object may be an object that a subject has not previously been exposed to. A novel object may be an object that a subject has previously investigated, probed, attended to, or examined, provided that the object at a present time invokes the subject's investigative, probing, attending or examining faculties. A novel object may be an object that the subject has not been exposed to for a period of at least about 1 hour, 2 hours, 4 hours, 6 hours, 8 hours, 10 hours, 12 hours, 18 hours, 24 hours, 36 hours, 48 hours or more, provided that the object at a present time invokes the subject's investigative, probing, attending or examining faculties.

It should be appreciated that an object that is familiar to a subject may become novel to the subject after a sufficient period of time has passed from a previous exposure to the object. In essence, a subject may “forget” having been exposed to an object in some cases. A subject's ability to perceive an object as being novel after having been previously exposed to the object will depend on a variety of factors, including, for example, the type of subject (e.g., species, genetic background, age, disease status, etc.), the type of object (e.g., color, shape, size, texture, etc.), time since last exposure to the object and the history of past exposure (e.g., duration of exposure, frequency of exposure, etc.).

An exploratory activity often involves a body portion of a subject entering a predetermined distance from an object to be explored (e.g., a predetermined distance from a mid-point of an object to be explored). However, in some cases, a body portion of a subject comes within a predetermined distance from an object, when the subject is not actually exploring the object. It is often the case that when a subject is not actually exploring an object, but its body portion comes in proximity to the object, the duration of time that the body portion remains in proximity to the object is less than the duration of time that would be observed if the subject was actually exploring the object. Accordingly, depending on the method used, the exploratory activity may involve a body portion of the subject entering a predetermined distance from the object, such that after entry into the predetermined distance, the body portion is maintained within the predetermined distance for a predetermined duration. In some embodiments, activity in which a subject is within the predetermined distance for less than the predetermined duration is non-exploratory activity. Therefore, by assessing the duration of a subject's presence within the predetermined distance from an object it is possible in some cases to distinguish non-exploratory activity (e.g., random presence near an object) from exploratory activity.

In some embodiments, the time of initiation of the exploratory activity is the time the body portion of the subject entered the predetermined distance. The body portion may be the subject's mid-torso, limb, finger, hand, foot, nose, paw, snout, or vibrissae, for example. The predetermined distance may be in a range of 0.01 times to 2 times the length of the subject, 0.05 times to 0.5 times the length of the subject, or 0.1 times to 0.3 times the length of the subject. The predetermined distance may be 0.01 times, 0.05 times, 0.1 times, 0.2 times, 0.3 times, 0.4 times, 0.5 times, 0.6 times, 0.7 times, 0.8 times, 0.9 times, 1 times, or 2 times the length of the subject. The predetermined distance may be in a range of 0.05 cm to 200 cm, 0.1 cm to 100 cm, 1 cm to 50 cm, or 1 cm to 10 cm. The predetermined distance may be 0.05 cm, 0.1 cm, 0.5 cm, 1 cm, 2 cm, 3 cm, 4 cm, 5 cm, 6 cm, 6.5 cm, 7 cm, 8 cm, 9 cm, 10 cm, 19 cm, 20 cm, 30 cm, 40 cm, 50 cm, 100 cm, or 200 cm. The predetermined duration may be up to 0.1 second, up to 0.25 second, up to 0.5 second, up to 1 second, up to 2 seconds, up to 5 seconds, up to 10 seconds, up to 30 seconds, up to 60 seconds, up to 120 seconds, up to 5 minutes, up to 10 minutes or more. The predetermined duration may be in a range of 0.1 second to 1 second, 0.5 second to 1 second, 0.5 second to 5 seconds, 1 second to 10 seconds, 1 second to 30 seconds, 1 second to 60 seconds, 1 second to 120 seconds, 5 seconds to 5 minutes, or 1 minute to 10 minutes.

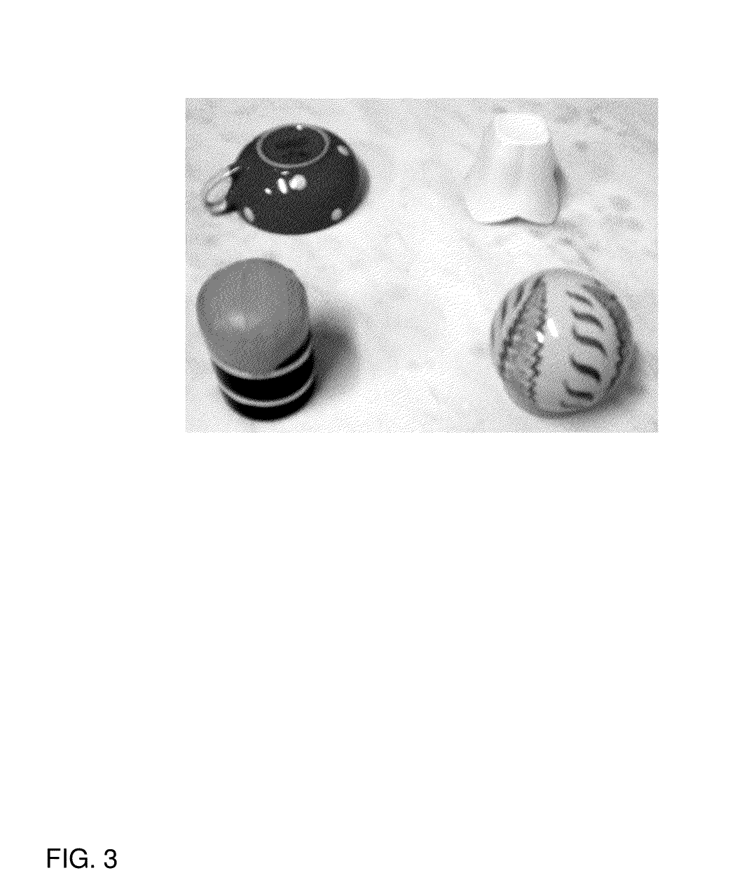

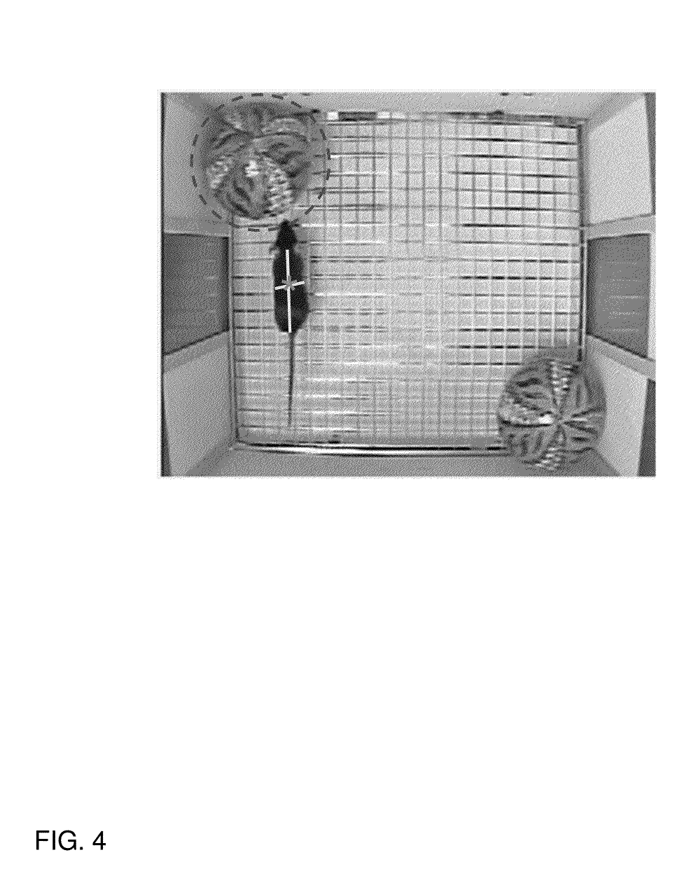

In some cases, a subject, or a body portion of a subject, may approach an object, and remain in proximity to the object, when the subject is not actually investigating, probing or examining the object. For example, a rodent may sit on an object randomly or for a purpose other than to explore the object. In some cases, the midpoint of a subject being within the predetermined distance from the object indicates that the subject is not exploring the object. It may be possible, in some cases, to identify this non-exploratory activity by examining the proximity of a plurality of different body portions of the subject relative to the object. For example, a rodent that is exploring an object may have its snout in proximity to the object relative to its mid-torso, such as would be the case if the rodent's longitudinal body-axis (medial body axis) was aligned approximately perpendicularly to an outer surface of the object and the rodent's snout was in proximity to the object relative to its mid-torso. Such an alignment is often characteristic of a rodent engaged in an exploratory activity in a novel object recognition task (See, e.g., FIG. 4). In contrast, when a rodent is sitting on an object, and not exploring the object, the relative position of its snout and mid-torso in relationship to the object may be different. For example, a rodent's mid-torso may be closer to the object, relative to its snout, when the rodent is sitting on the object. Accordingly, detecting differences in the relative positions of body portions of a subject may provide a basis for discriminating exploratory activity from non-exploratory activity in some cases.

In some cases, it may be possible to discriminate exploratory activity from non-exploratory activity by evaluating a subject's head or body orientation relative to an object. For example, a subject engaging in an exploratory activity may have a medial head axis orientated within a range of 0° to 5°, 0° to 20°, 0° to 30°, 0° to 45°, or 0° to 60° from an axis passing through the center of the object and the center of the subject's head.

An exploratory activity may involve probing, investigating, attending and/or examining an object for a duration of time of up to 10 seconds, up to 20 seconds, up to 30 seconds, up to 40 seconds, up to 50 seconds, up to 60 seconds, up to 90 seconds, up to 180 seconds, up to 5 minutes, up to 10 minutes, up to 20 minutes, or up to 30 minutes. An exploratory activity may involve probing, investigating and/or examining an object for a duration of time in a range of between 1 second and 10 seconds, between 5 seconds and 30 seconds, between 10 seconds and 60 seconds, between 30 seconds and 90 seconds, between 60 seconds and 180 seconds, between 1 minute and 5 minutes, between 1 minute and 10 minutes, or between 5 minutes and 30 minutes. An exploratory activity may involve probing, investigating and/or examining an object for a duration of time of about 10 seconds, about 20 seconds, about 30 seconds, about 40 seconds, about 50 seconds, about 60 seconds, about 90 seconds, about 180 seconds, about 5 minutes, about 10 minutes, about 20 minutes, about 30 minutes, or more.

In methods where exposing the subject to the object comprises presenting an image of the object in the perceptual environment of the subject, the exploratory activity often comprises the subject's gaze being focused on the object for a predetermined duration. An illustrative example of such a method is disclosed in Courchesne, E., S. A. Hillyard, and R. Galambos, Stimulus novelty, task relevance and the visual evoked potential in man. Electroencephalogr Clin Neurophysiol, 1975. 39(2): p. 131-43. In some cases, the subject may make an affirmative indication that the subject is engaging in an exploratory activity. For example, in some cases a subject may push a button, pull a lever, click a mouse, recite a sound, etc. as an affirmative indication that the subject is engaging in an exploratory activity.

In some embodiments, the time of initiation of the exploratory activity relating to an image occurs when the subject's gaze begins focusing on the object for the predetermined duration. The predetermined duration may be continuous or cumulative. The predetermined duration may be up to 0.01 second, up to 0.1 second, up to 0.5 second, up to 1 second, up to 2 seconds, up to 5 seconds, up to 2 seconds, up to 30 seconds, up to 60 seconds, up to 120 seconds, up to 5 minutes, up to 10 minutes or more. The predetermined duration may be in a range of 0.1 second to 1 second, 0.5 second to 1 second, 0.5 second to 5 seconds, 1 second to 10 seconds, 1 second to 30 seconds, 1 second to 60 seconds, 1 second to 120 seconds, 5 seconds to 5 minutes, or 1 minute to 10 minutes.

As used herein, the term “subject” refers to any animal. The subject may be, for example, a rodent, a cat, a dog, a primate or any other suitable animal. In some embodiments, a rodent is a rat or a mouse. In some embodiments, a primate is a non-human primate and in some embodiments, a primate is a human. The subject may be a model of a disease, disorder, deficit or condition. The subject may be afflicted by a disease, disorder, deficit or condition. For example, the subject may be a human subject who is afflicted by a disease, disorder, deficit or condition.

### Electroencephalographic Oscillations

In some embodiments, methods provided herein involve inducing a subject to initiate an exploratory activity and recording electroencephalographic oscillations from the subject. As used herein the term “electroencephalographic oscillations” refers to electrophysiological signals that are recorded from the brain of a subject. Electroencephalographic oscillations may be equivalently referred to herein as “EEG oscillations,” “electroencephalographic signals,” or “EEG signals.” As used herein, the term “record” means to acquire, to obtain, to observe, and/or to store. An electroencephalographic oscillation may be recorded as a time-dependent voltage between a pair of electrodes positioned on, or in proximity to, brain tissue and recorded over a discrete period of time. These electroencephalographic oscillations may be acquired by an electroencephalogram device, e.g., a system that can measure an electrical activity in the brain via one or more internal or external electrode configurations. For example, electrodes may be coupled to a subject's scalp or implanted in the subject's brain tissue to acquire electroencephalographic oscillations.

A variety of methods are known in the art for acquiring EEG oscillations from a subject. Such methods can be used to acquire EEG oscillations during onset of an exploratory activity, according to the methods disclosed herein. Non-limiting examples of art-known methods for acquiring EEG oscillations are disclosed in J. Martinovic et al., Induced gamma-band activity is related to the time point of object identification, Brain Research 1198 (2008) 93-106; G. Stefanics et al., EEG Early Evoked Gamma-Band Synchronization Reflects Object Recognition in Visual Oddball Tasks, Brain Topography 16(4) (2004) 261-264; J. R. Clarke et al., Plastic modifications induced by object recognition memory processing, Proceeding of the National Academy of Sciences USA 107(6) (2010) 2652-2657; T. Curran et al., An electrophysiological comparison of visual categorization and recognition memory, Cognitive, Affective, & Behavior Neuroscience, 2(1) (2002) 1-18; A. Sambeth et al., Cholinergic drugs affect novel object recognition in rats: Relation with hippocampal EEG?, European Journal of Pharmacology 572 (2007) 151-159; E. L. Mazerolle et al., ERP assessment of functional status in the temporal lobe: Examining spatiotemporal correlates of object recognition, International Journal of Psychophysiology 66 (2007) 81-92; J. D. Harris et al., Neurophysiological indices of perceptual object priming in the absence of explicit recognition memory, International Journal of Psychophysiology 71 (2009) 132-141; K. A. Snyder et al., Repetition Suppression of Induced Gamma Activity Predicts Enhanced Orienting toward a Novel Stimulus in 6-month-old Infants, Journal of Cognitive Neuroscience 20(12) (2008) 2137-2152; J. R. Manns et al., Hippocampal CA1 spiking during encoding and retrieval: Relation to theta phase, Neurobiology of Learning and Memory 87 (2007) 9-20; M. J. Gandal et al., A Novel Electrophysiological Model Of Chemotherapy-Induced Cognitive Impairments In Mice, Neuroscience 157 (2008) 95-104; and E. C. Leek et al., Computational mechanisms of object constancy for visual recognition revealed by event-related potentials, Vision Research 47 (2007) 706-713.

Some aspects of the invention include stereotaxic implantation of microwire bundle electrodes into the prefrontal cortex (PFC) of subjects. The location of the implantation may be in a region of brain that is within medial-lateral extent posterior to the olfactory bulb, anterior to M2 motor cortex, and superficial to orbital cortex. Exemplary, but non-limiting implantation coordinates in mice include: from Bregma: +0.37 cm rostral, +0.07 cm lateral, and −0.05 cm deep from brain surface. Following implantation, and after a recovery period for the subject, EEG traces from PFC can be recorded from the freely behaving subject during a pre-initiation period and/or during an exploratory activity. Appropriate coordinates of other subjects will be known by those skilled in the art. Appropriate coordinates for stereotaxic implantation of microwire bundle electrodes to areas of a subject's brain other than the PFC will also be known by those skilled in the art. In some aspects the invention includes stereotaxic implantation of microwire bundle electrodes to multiple areas of a subject's brain.

The invention, in some aspects, provides methods for recording EEG oscillations in a PFC region of a subject engaged in a task that involves an exploratory activity. In some embodiments, single unit activity (SUA) may be recorded from an implanted electrode. In some embodiments, recording may be performed using scalp electrodes or other non-invasive recording electrodes or devices. As provided herein, EEG oscillations may be acquired after the subject has been administered a test agent or candidate therapeutic agent. Furthermore, the EEG oscillations acquired from the subject may be compared to EEG oscillations of a control subject, or the EEG oscillations acquired from the test subject prior to administration of the agent, to identify whether the agent modulates EEG oscillations, e.g., whether the agent effects the presence or absence of a particular signature in EEG oscillations during onset of an exploratory activity. In some embodiments, treatment of the subject with the agent results the particular signature in EEG oscillations being present during onset of an exploratory activity. In some embodiments, treatment of the subject with the agent results the particular signature in EEG oscillations being absent during onset of an exploratory activity.

Electroencephalographic oscillations may be processed (e.g., band-pass filtered, etc.) to obtain a component oscillation having a desired frequency (e.g., a frequency in a range of 30 Hz to 90 Hz, a frequency in a range of 65 Hz to 90 Hz, etc.). For example, to quantify gamma oscillations, recordings of electroencephalographic oscillations may be band-pass filtered to obtain oscillations having a frequency range of 1 Hz to 5 Hz, 5 Hz to 10 Hz, 10 Hz to 20 Hz, 20 Hz to 30 Hz, 30 Hz to 90 Hz, 30 Hz to 55 Hz, 65 Hz to 90 Hz, 65 Hz to less than 100 Hz, etc. In some embodiments, electroencephalographic oscillations are up to 1 Hz, 5 Hz, 10 Hz, 20 Hz, 30 Hz, 40 Hz, 50 Hz, 60 Hz, 70 Hz, 80 Hz, 90 Hz, 100 Hz, 150 Hz, 200 Hz, 250 Hz, 300 Hz, 350 Hz, 400 Hz, 450 Hz, 500 Hz, 750 Hz, 1000 Hz, 1500 Hz or more Hz including all values in between. In some embodiments, electroencephalographic oscillations are in a range of about 1 Hz to 5 Hz, 5 Hz to 10 Hz, 10 Hz to 20 Hz, 20 Hz to 30 Hz, 30 Hz to 40 Hz, 40 Hz to 50 Hz, 50 Hz to 60 Hz, 60 Hz to 70 Hz, 70 Hz to 80 Hz, 80 Hz to 90 Hz, 90 Hz to 100 Hz, 100 Hz to 150 Hz, 150 Hz to 200 Hz, 200 Hz to 250 Hz, 250 Hz to 300 Hz, 300 Hz to 350 Hz, 350 Hz to 400 Hz, 400 Hz to 450 Hz, 450 Hz to 500 Hz, 500 Hz to 750 Hz, 750 Hz to 1000 Hz, or 1000 Hz to 1500 Hz. In some embodiments, electroencephalographic oscillations are theta oscillations, beta oscillations, gamma oscillations, or ripple oscillations. Theta oscillations may have a frequency range of 4 Hz to 12 Hz or 4 Hz to 9 Hz. Beta oscillations may have a frequency range of 15 Hz to 30 Hz. Gamma oscillations may have a range of 30 Hz to 90 Hz. Gamma oscillations may have a range of 30 Hz to up to 100 Hz. Ripple oscillations may have a range of 100 Hz to 300 Hz.

It will be understood that an electroencephalographic oscillation may be represented or displayed in any one of a variety of ways. For example, the electroencephalographic oscillation, may be represented in a time domain, e.g., as a voltage time series or as a power time series. The electroencephalographic oscillation may be represented in a frequency domain, e.g., by transforming a signal from a time domain to a frequency domain (e.g., using Fast-Fourier Transform, Wavelet Transform, etc.). It should also be understood that a recording of an electroencephalographic oscillation may be processed in any one of a variety of ways to quantify different oscillatory components of the signal. In some embodiments, electroencephalographic oscillations may be represented as the frequency of occurrence of power (or voltage) levels in the oscillation.

### Signatures in EEG Oscillations

Methods are provided herein for determining the presence or absence of signatures in electroencephalographic oscillations recorded from a subject during onset of an exploratory activity. As used herein, the phrase, “signature in electroencephalographic oscillations” refers to a distinctive characteristic of an electroencephalographic oscillation that is indicative of the cognitive status of a subject. In some embodiments, presence of the signature is indicative of absence of a cognitive disorder. In some embodiments, absence of the signature is indicative of presence of a cognitive disorder. In some embodiments, presence of the signature is indicative of presence of a cognitive disorder. In some embodiments, absence of the signature is indicative of absence of a cognitive disorder.

In some embodiments, a signature is based at least in part on the power of electroencephalographic oscillations. In some embodiments, the signature is based at least in part on evoked power of electroencephalographic oscillations. In some embodiments, the signature is based at least in part on the power in one or more frequency bands of the electroencephalographic oscillations. Accordingly, in some embodiments, determining the presence or absence of a signature in electroencephalographic oscillations involves performing a power spectral analysis of the electroencephalographic oscillations. In some embodiments, determining the presence or absence of a signature in electroencephalographic oscillations involves performing a spectral decomposition of the electroencephalographic oscillations. In some embodiments, the signature is based at least in part on one or more phase locking characteristics of electroencephalographic oscillations. In some embodiments, a signature of EEG oscillations may comprise information from statistical time-frequency maps, for example, time-frequency maps of p-values that reflect the significance of observed differences between power values or phase locking factors between two conditions, e.g., a novel image stimulus compared with a dim image stimulus. It should be appreciated that the presence or absence of a signature in an electroencephalographic oscillations is generally determined through the use of a computer encoded with instructions for processing data representative of electroencephalographic oscillations recorded from a subject.

### EEG Power

Power of electroencephalographic oscillation may be evaluated or determined by any one of a variety of methods known in the art. In some embodiments, the power is determined by processing the electroencephalographic oscillations using a spectral analysis. Spectral analysis methods that may be applied in conjunction with methods disclosed herein for use to analyze electroencephalographic oscillations are well known in the art (See, e.g., Van Vugt M. K. et al., Comparison of Spectral Analysis Methods for Characterizing Brain Oscillations, Journal of Neuroscience Methods, (2007) 162:49-63; Klimesch W. et al., Episodic and semantic memory; an analysis in the EEG theta band, Electroencephalogr Clin Neurophysiol 1994; 91:428-41; Whittington M. A. et al., Inhibition-based rhythms: experimental and mathematical observations on network dynamics, Int J Psychophysiol, (2000) 38:315-336; Spencer K. M. et al., Sensory-evoked gamma oscillations in chronic schizophrenia, Biol Psychiatry, (2008) 63:744-747; the contents of which relating to spectral analysis of electroencephalographic signals are incorporated herein by reference).

In some embodiments, EEG power is determined by frequency decomposition of the EEG oscillations. A fast-fourier transform (FFT) may be used to spectrally decompose EEG oscillations. This may result in a power spectrum that captures the average magnitude of oscillations for individual frequency bins integrated over a particular time period. The frequency resolution is determined, at least in part, by the number of time points contains in the time windows (e.g., which can be determined by multiplying the sampling rate by the sampling duration.)

In some embodiments, event-related power of EEG oscillations is determined. Event-related power may be determined by squaring the magnitude of a vector obtained from spectral decomposition of EEG oscillations, on a 2-dimensional, real-imaginary plane. In such embodiments, event-related power reflects the magnitude of EEG oscillations at specific frequencies.

In some embodiments, the powers levels (e.g., event-related power, power obtained by FFT) are used to produce a 2-dimensional matrix contain the power of the EEG oscillations at each frequency and time point (a time-frequency matrix of power values). In some embodiments, the powers levels (e.g., event-related power, power obtained by FFT) are averaged over a series of trials or experiments to produce a 2-dimensional matrix contain the average power of the EEG oscillations at each frequency and time point.

It should be appreciated that total power captures the magnitude of oscillations independent of their phase angles. Thus, the total power comprises both induced power and evoked power. Induced power refers to event-related changes in EEG power that are time-locked, but not phase-locked, with respect to the event onset across trials and/or subjects. Evoked power refers to event-related changes in EEG power that are phase-locked with respect to an event onset (e.g., initiation of an exploratory activity) across trials and/or subjects. In some embodiments, phase-locked oscillations may be isolated by averaging (e.g., in the time domain) event-locked EEG epochs to derive event-related potentials. Frequencies that are phase synchronized with respect to stimulus onset across repeated trials and/or subjects survive the averaging process and can be detected in the average event-related potentials. Thus, in some embodiments, evoked power may be determined by performing a spectral decomposition of event related potentials, and squaring the magnitude values associated with each time and frequency point in a time-frequency matrix.

### Power Spectral Density

In some embodiments, power may be determined from a power spectral density (PSD) that measures power per unit of frequency in an EEG oscillation. Any one of a variety of different methods may be used to determine the power spectral density of the EEG oscillation, or segment thereof, including, for example, nonparametric and parametric methods. Nonparametric methods are typically those in which the PSD is estimated directly from the EEG oscillation. An example of such a method is the periodogram. Other nonparametric techniques include, but are not limited to, Welch's method and the multitaper method (MTM) both of which may reduce the variance of the periodogram. Parametric methods are those in which the PSD is estimated from a signal that is assumed to be an output of a linear system driven by white noise. Non-limiting examples of parametric methods are the Yule-Walker autoregressive (AR) method and the Burg method. Those skilled in the art will be aware of additional parametric and non-parametric methods that may be used in methods of the invention.

Power may be determined from an EEG oscillation as the maximum value of the PSD within a predetermined frequency range. Alternatively, power may be determined from an EEG oscillation as the area under the curve of the PSD function within a predetermined frequency range. The area under the curve of the PSD function may be obtained by integrating the PSD function (e.g., using trapezoidal numerical integration) across a predetermined frequency range. Power obtained using the area under the curve approach may be referred to herein as “ensemble EEG power.” The skilled artisan will appreciate that still other alternative methods for determining the power of an EEG oscillation may be used. For example, the arithmetic mean of the PSD function within a predetermined frequency range, the median of the PSD function with the predetermined frequency range, etc. In some embodiments, the power of an EEG oscillation is determined in the time domain. For example, the power may be estimated as the root mean square of an EEG oscillation that is a voltage time series, which may be a band-pass filtered voltage time series.

The predetermined frequency range of the PSD, from which the power of an EEG oscillation is determined, may be a frequency range corresponding to a gamma oscillation (e.g., 30 Hz to 90 Hz). In some embodiments, the predetermined frequency range corresponds to the upper portion of the gamma oscillation range (e.g., 65 Hz to 90 Hz, 65 Hz to 100 Hz). Other appropriate frequency ranges are disclosed herein and will be apparent to the skilled artisan.

In some embodiments of the invention, a power distribution is determined by obtaining data of EEG oscillations obtained from subjects over a continuous recording session and performing power analyses on consecutive time segments of the data. Data may be binned into any of a variety of time segments, for example, 0.5 sec., 1 sec., 5 sec., 10 sec., 15 sec., 20 sec., 25 sec., 30 sec. segments (including all times in between), and analyzed. A relative frequency histogram (a distribution) may be constructed by binning powers determined for each time segment over an entire recording session. A non-limiting example of computing the distribution of power includes taking data from individual subjects obtained over a continuous recording session (e.g., a 2 minute, a 5 minute, a 10 minute, or a 30 minute recording session) and performing power analyses on consecutive segments (e.g., 0.5 second, 1 second, 5 second, 10 second, 20 second, or 30 second segments). A relative frequency histogram is constructed from binning the ensemble powers for each segment over an entire recording session. Alternative time periods for recording sessions and binned segments may be used in methods of the invention.

### EEG Phase Locking Characteristics

In some embodiments, the extent to which phase locking occurs with respect to initiation of an exploratory activity may be determined. To accomplish this a phase locking factor may be determined by averaging a normalized complex power of EEG oscillations across trials. This results in a value (i.e., the phase-locking factor (PLF)) that describes the phase distribution in a time-frequency domain. The PLF ranges from 0 to 1, with 0 indicating non-phase locked activity, and 1 indicating strictly phase locked activity. In some embodiments, a signature of electroencephalographic oscillations is based on a phase locking factor.

### Process for Assessing Signatures in EEG Oscillations

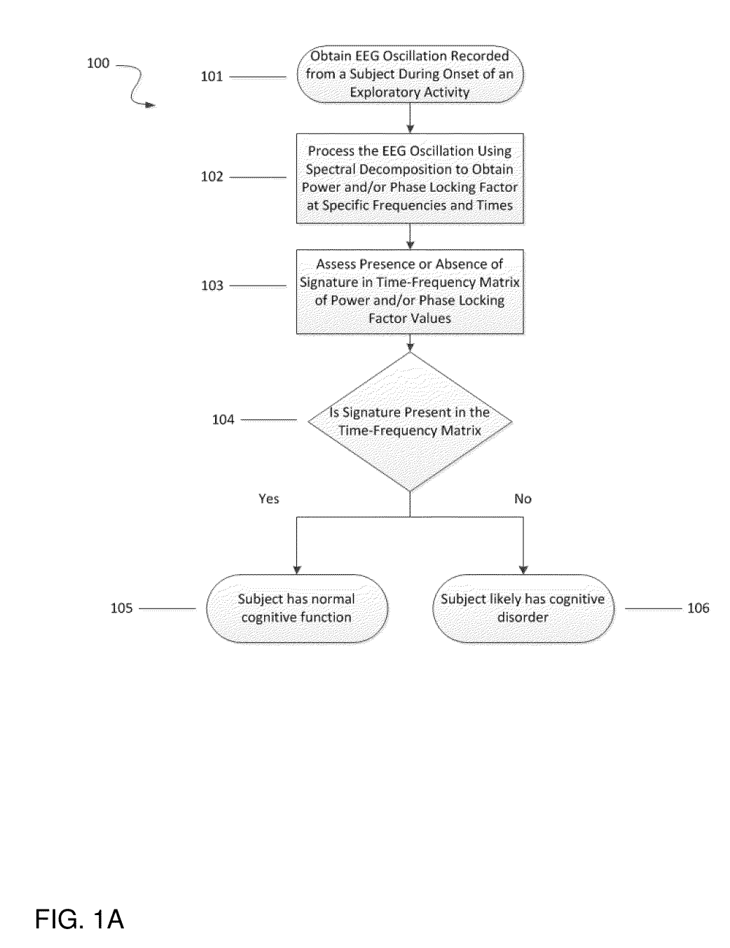

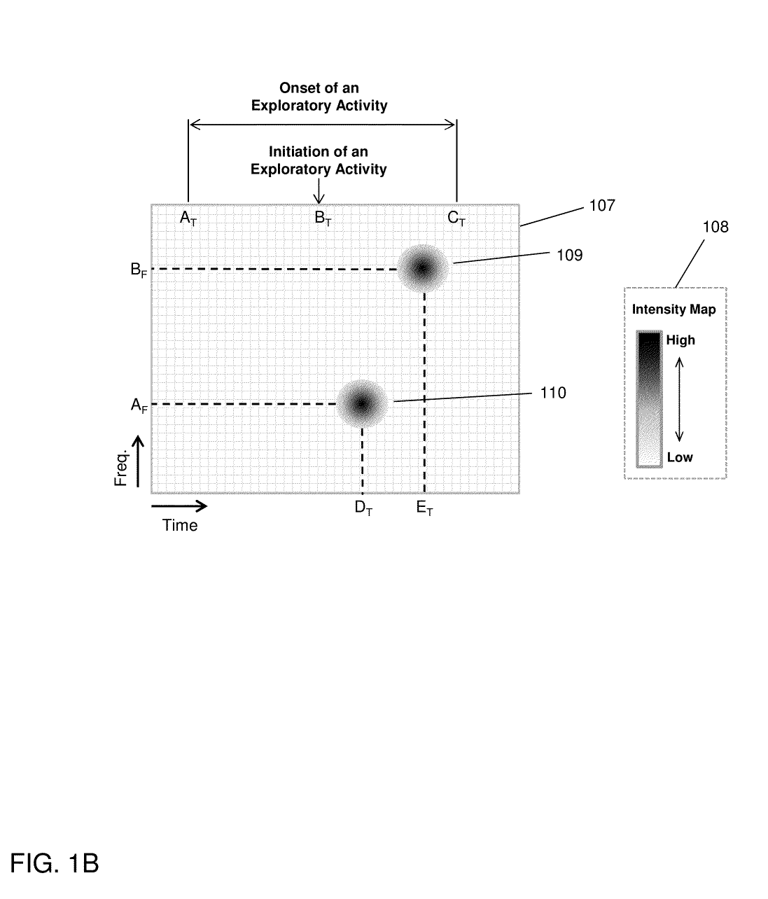

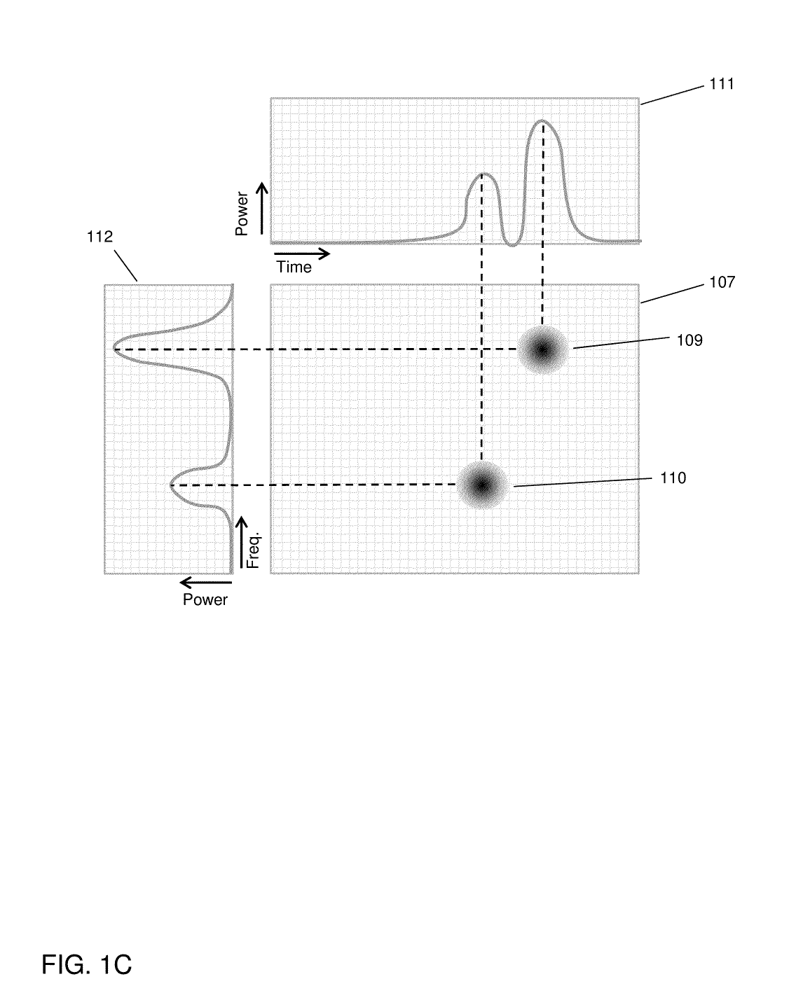

FIG. 1A illustrates an exemplary process 100 for detecting a signature in electroencephalographic oscillations. At start block 101, an EEG oscillation is obtained. The EEG oscillation is recorded from a subject during onset of an exploratory activity (e.g., a time period encompassing onset of the exploratory activity in the subject). The EEG oscillation may be recorded, for example, using an implanted electrode, or an implanted bundle of electrodes. External electrodes (e.g., scalp electrodes) or other non-invasive electrodes, may alternatively be used to obtain an EEG oscillation from the subject. At block 102, the EEG oscillation is processed (e.g., using spectral decomposition) to obtain power values or phase locking factors at specific frequencies and times, thereby obtaining a time-frequency matrix of power values or phase locking factors. At block 103, the power values or phase locking factors are assessed within the time-frequency space to detect the presence or absence of a signature indicative of cognitive function in the subject. The signature may comprise power information, phase locking information or a combination thereof. At decision block 104, the presence of the signature in the time-frequency space indicates that the subject has normal cognitive function, as indicated at end block 105; whereas the absence of the signature in the time-frequency space indicates that the subject is likely to have a cognitive disorder, as indicated at end block 106.

In some embodiments, an EEG oscillation is processed using spectral decomposition. The EEG oscillation information is mapped to real and imaginary components for each time point and at each frequency within a range of frequencies (e.g., 1 Hz to 300 Hz, 10 Hz to 100 Hz). In some embodiments, the phase angles from the complex numbers are removed, and the remaining magnitude values are squared and then averaged, providing an estimate of total power at each particular frequency and time. In some embodiments, the magnitude values are removed from the complex numbers, and the remaining equal length vectors, which retain phase angle information, are averaged to obtain the phase-locking factor (PLF). Repeating these steps for each time and frequency point of an EEG oscillation record yields a time-frequency matrices of total power values and PLFs.

Referring to FIG. 1B, an illustration of an time-frequency map 107 is provided, in which the intensity of power values are plotted in time-frequency coordinates. A similar map may be produced from other characteristic measures of EEG oscillations, e.g., phase locking factor. A gray-scale intensity map 108 is provided with high power values tending towards black, and low values tending towards white. The time scale on the time-frequency map 107 encompasses the onset of an exploratory activity which begins at time, A_{T}, and extends to time, C_{T}. Within this time period, initiation of the exploratory activity occurs at time, B_{T}. Two maxima are indicated in the time-frequency map 107. A first maxima 110 is centered at time, D_{T}, and frequency, A_{F}. A second maxima 109 is centered at time, E_{T}, and frequency, B_{F}. For illustrative purposes, the maxima 109, 110 are depicted in FIG. 1C as projections in power-time coordinates in graph 111 and in power-frequency coordinates in graph 112. In this embodiment, the signature of EEG oscillations during onset of the exploratory activity comprises power maxima at time-frequency coordinates (D_{T}, A_{F}) and (E_{T}, B_{F}). If, for example, this signature represented that of a subject who does not have a cognitive disorder, then the signature may serve as a biomarker for normal cognitive function. A graph of power values in time-frequency coordinates produced from EEG oscillations obtained from a subject who has a cognitive disorder may lack one or both of the maxima 109, 110, and thus, exhibit an absence of the signature observed in the normal subject. In this case, the biomarker serves as a basis for objectively discriminating between normal cognitive function and abnormal cognitive function. It should be appreciated that a signature could also be produced based on phase locking factor values by assessing the presence or absence of phase-locking factor maxima or minima in time-frequency coordinates. Maxima may be referred to herein as peaks.

Accordingly, it should be appreciated, that methods are provided herein for determining the presence or absence of signatures in electroencephalographic oscillations recorded from a subject. The signatures in electroencephalographic oscillations may manifest as a set of distinctive characteristics in the power of electroencephalographic oscillations (or in another EEG oscillation characteristic, e.g., phase-locking factor) that is indicative of the cognitive status of a subject. In some embodiments, the signature comprises a set of maxima and/or minima in a time-frequency matrix of the power or phase locking factors of EEG oscillations. Thus, the signature may comprise the approximate position in time-frequency coordinates of local maxima and/or local minima present in a time-frequency matrix of power or phase locking factor determined from EEG oscillations. In some embodiments, the power values are absolute values. In some embodiment, the power values are standardized across the time-frequency matrix. In some embodiments, the power values are normalized. For example, the power values may be normalized to a baseline (e.g., an untreated condition, a control subject, etc.) In some embodiments, the phase locking factors are absolute values (e.g., ranging from 0 to 1, with 0 indicting no phase locking, and 1 indicating strict phase-locking). In some embodiment, the phase locking factors are standardized across the time-frequency matrix. In some embodiments, the phase locking factors are normalized. For example, the phase locking factors may be normalized to a baseline (e.g., an untreated condition, a control subject, etc.)

### Test Conditions for an Exploratory Activity

It should be appreciated that a variety of different experimental or test conditions may be used to evaluate in a subject during onset of an exploratory activity. For example, in cases where an exploratory activity, which involves a subject exploring a physical object, is identified by a subject's body portion entering within a predetermined distance from the object for a predetermined duration, the predetermined distance and predetermined duration may vary depending on a variety of factors including, for example, the subject, the object and the environment within which the subject is engaging in the exploratory activity. Moreover, a variety of different conditions may be used to identify a signature in encephalographic oscillations. The duration of onset of an exploratory activity, for example, may vary. The skilled artisan will be capable of selecting an appropriate set of experimental or test conditions. Tables 1 and 2 provide exemplary conditions for some embodiments of the invention. These conditions are appropriate for experiments or tests in which an exploratory activity involves a subject exploring a physical object, such as, for example, a novel objection recognition test.

Table 1 outlines forty (X1-X40) examples of test conditions that may be used for an exploratory activity. For example, for test condition X1 an exploratory activity involves a body portion of a subject being within a distance from an object of up to 0.01 times the body length of the subject and remaining within that distance for at least up to 0.05 seconds. When these conditions are satisfied the exploratory activity is identified, and initiation of the exploratory activity is determined as the time when the subject first entered within the distance from the object of up to 0.01 times the body length of the subject.

TABLE 1
Examples of test conditions for identifying an exploratory activity.

| | | Magnitude of Predetermined Distance | | | | |
|---|---|---|---|---|---|---|
| | | (Body Lengths of a Subject) | | | | |
| | | up to 0.01 | 0.01 to 0.05 | 0.05 to 0.1 | 0.1 to 0.5 | 0.5 to 2 |
| Duration Within | up to 0.05 | X1 | X9 | X17 | X25 | X33 |
| Predetermineed Distance (sec.) | 0.05 to 0.1 | X2 | X10 | X18 | X26 | X34 |
| | 0.1 to 0.2 | X3 | X11 | X19 | X27 | X35 |
| | 0.2 to 0.5 | X4 | X12 | X20 | X28 | X36 |
| | 0.5 to 1 | X5 | X13 | X21 | X29 | X37 |
| | 1 to 2 | X6 | X14 | X22 | X30 | X38 |
| | 2 to 5 | X7 | X15 | X23 | X31 | X39 |
| | 5 to 10 | X8 | X16 | X24 | X32 | X40 |

Table 2 outlines one-hundred eighty (Y1-Y180) examples of test conditions for evaluating EEG oscillations that occur during onset of an exploratory activity. The table outlines, for each of X1-X40 test conditions, five examples of onset periods within which a signature of EEG oscillations may be detected. As an example, for test condition X1 the table specifies five onset periods that may be used, which correspond to Y1, Y41, Y81, Y101, and Y141. At Y1, for example, a onset period of 4 sec prior to initiation to 1 sec after initiation is specified. According to Y1, EEG oscillations recorded from a subject (who engages in an exploratory activity having the test conditions of X1) will encompass an onset period of 4 sec prior to initiation of an exploratory activity to 1 sec after initiation, and the presence or absence of the signature may be assessed within that onset period.

TABLE 2
Examples of test conditions for evaluating a EEG oscillations that occur during onset
of an exploratory activity

| | | Duration of Onset Period (sec.) | | | | |
|---|---|---|---|---|---|---|
| | | | | | 3 sec prior | 2 sec prior |
| | | 4 sec prior | 3 sec prior | 2 sec prior | to | to |
| | | to initiation | to initiation | to initiation | initiation | initiation |
| | | to 1 sec | to 1 sec | to 1 sec | to the | to the |
| | | after | after | after | point of | point of |
| | | initiation | initiation | initiation | initiation | initiation |
| Exploratory Activity Design Conditions | X1 | Y1 | Y41 | Y81 | Y101 | Y141 |
| | X2 | Y2 | Y42 | Y82 | Y102 | Y142 |
| | X3 | Y3 | Y43 | Y83 | Y103 | Y143 |
| | X4 | Y4 | Y44 | Y84 | Y104 | Y144 |
| | X5 | Y5 | Y45 | Y85 | Y105 | Y145 |
| | X6 | Y6 | Y46 | Y86 | Y106 | Y146 |
| | X7 | Y7 | Y47 | Y87 | Y107 | Y147 |
| | X8 | Y8 | Y48 | Y88 | Y108 | Y148 |
| | X9 | Y9 | Y49 | Y89 | Y109 | Y149 |
| | X10 | Y10 | Y50 | Y90 | Y110 | Y150 |
| | X11 | Y11 | Y51 | Y91 | Y111 | Y151 |
| | X12 | Y12 | Y52 | Y92 | Y112 | Y152 |
| | X13 | Y13 | Y53 | Y93 | Y113 | Y153 |
| | X14 | Y14 | Y54 | Y94 | Y114 | Y154 |
| | X15 | Y15 | Y55 | Y95 | Y115 | Y155 |
| | X16 | Y16 | Y56 | Y96 | Y116 | Y156 |
| | X17 | Y17 | Y57 | Y97 | Y117 | Y157 |
| | X18 | Y18 | Y58 | Y98 | Y118 | Y158 |
| | X19 | Y19 | Y59 | Y99 | Y119 | Y159 |
| | X20 | Y20 | Y60 | Y100 | Y120 | Y160 |
| | X21 | Y21 | Y61 | Y101 | Y121 | Y161 |
| | X22 | Y22 | Y62 | Y102 | Y122 | Y162 |
| | X23 | Y23 | Y63 | Y103 | Y123 | Y163 |
| | X24 | Y24 | Y64 | Y104 | Y124 | Y164 |
| | X25 | Y25 | Y65 | Y105 | Y125 | Y165 |
| | X26 | Y26 | Y66 | Y106 | Y126 | Y166 |
| | X27 | Y27 | Y67 | Y107 | Y127 | Y167 |
| | X28 | Y28 | Y68 | Y108 | Y128 | Y168 |
| | X29 | Y29 | Y69 | Y109 | Y129 | Y169 |
| | X30 | Y30 | Y70 | Y110 | Y130 | Y170 |
| | X31 | Y31 | Y71 | Y111 | Y131 | Y171 |
| | X32 | Y32 | Y72 | Y112 | Y132 | Y172 |
| | X33 | Y33 | Y73 | Y113 | Y133 | Y173 |
| | X34 | Y34 | Y74 | Y114 | Y134 | Y174 |
| | X35 | Y35 | Y75 | Y115 | Y135 | Y175 |
| | X36 | Y36 | Y76 | Y116 | Y136 | Y176 |
| | X37 | Y37 | Y77 | Y117 | Y137 | Y177 |
| | X38 | Y38 | Y78 | Y118 | Y138 | Y178 |
| | X39 | Y39 | Y79 | Y119 | Y139 | Y179 |
| | X40 | Y40 | Y80 | Y120 | Y140 | Y180 |

### Novel Object Recognition Task

A novel object recognition (NOR) task is an example of a method suitable for evaluating EEG oscillations recorded during onset of an exploratory activity. In the novel object recognition task, initiation of an exploratory activity occurs when at least a portion of the subject's body (i.e., a body portion of the subject) enters within a predetermined distance from an object and remains within that predetermined distance for a predetermined period of time. In the NOR task, the exploratory activity involves a body portion (e.g., a nose) of a subject (e.g., a rodent) entering a predetermined distance from an object, such that after entry into the predetermined distance, the body portion is maintained within the predetermined distance for a predetermined duration. The time of initiation of the exploratory activity is the time the body portion of the subject entered the predetermined distance.

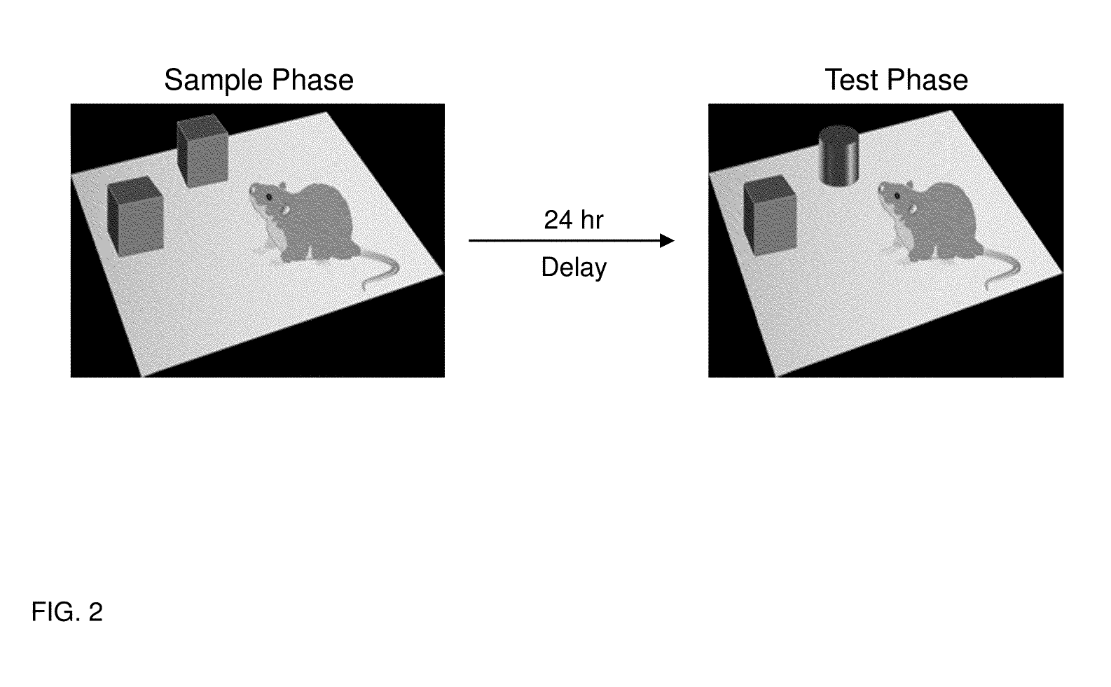

The NOR task is based on a tendency of a subject to preferentially investigate a novel object versus a familiar one. The choice to explore the novel object is understood to reflect the use of cognitive processes, such as, for example, attention, learning or memory. A typical novel object recognition task involves at least two phases. In the first phase, a subject (e.g., rodent) is positioned in an enclosure. Two or more substantially identical objects are also positioned in the enclosure within the perceptual environment of the subject. Typically, the two or more objects are located a specified distance from each other within the enclosure. In the second phase, the subject is positioned in an enclosure and two or more objects are also positioned in the enclosure within the perceptual environment of the subject, and at least one of the objects is an object that the subject was exposed to in the first phase (a familiar object) and at least one of the objects is a novel object. During the second phase, normal subjects have a tendency to investigate the novel object to a greater extent (e.g., for a greater duration) than the familiar object. A non-limiting example of an experimental configuration for a NOR task is depicted in FIG. 2. Moreover, examples of objects that may be used in the NOR are depicted in FIG. 3. For NOR task involving rodents, typically the objects are comparable in size to the rodent. Non-limiting examples of suitable objects include balls, cups, pens, markers, tape rolls, yarn balls, plastic toys, etc. Often the objects are of a weight that is sufficient to make them difficult for a rodent to move. In some cases, the objects are attached to an immobile surface (e.g., a floor) to prevent movement.

During the first phase of the NOR task the subject typically engages in an exploratory activity that involves investigating, probing and/or examining the two or more objects. Following this exploratory activity the subject is typically familiar with the objects, such that upon a subsequent exposure to the objects, the subject does not typically engage in the same degree of exploratory activity (e.g., spends less time investigating, probing and/or examining the objects), if any exploratory activity is engaged in at all.

The first phase may be referred to as the sample phase. The first phase may have a duration of up to 1 minute, 2 minutes, 5 minutes, 10 minutes, 20 minutes or more total time (including time engaged in an exploratory activity and time not engaged in an exploratory activity). Alternatively, the first phase may have a duration such that for up to 10 seconds, up to 20 seconds, up to 30 seconds, up to 40 seconds, up to 50 seconds, up to 60 seconds, up to 90 seconds, up to 180 seconds, or more total time the subject is engaged in an exploratory activity (e.g., probing, investigating and/or examining an object).

Following the first phase, the subject is removed from the enclosure and a predetermined amount of time (e.g., 0.5 hour, 1 hour, 4 hours, 12 hours, 24 hours, 36 hours, 48 hours, etc.) is allowed to pass. In the second phase, the subject is positioned in the same enclosure (or a substantially identical enclosure). Two or more objects are also positioned in the enclosure within the perceptual environment of the subject, at least one of the objects being an object from the first phase (a “familiar” object), and at least one of the objects being an object that is different in appearance (e.g., shape, texture and/or color) than the objects from the first phase. Objects that differ in appearance (e.g., shape, texture and/or color) than the objects from the first phase are often used as novel objects.

During the second phase of the NOR task, the subject is typically exposed to the two or more objects, including at least one novel object and at least one familiar object, for a period of time during which the subject engages in an exploratory activity that typically involves investigating, probing and/or examining at least one object. For normal subjects, this activity is typically biased towards the novel objects, such that more time is spent investigating, probing and/or examining the novel objects than the familiar objects. The second phase may be referred to as the test phase.

The second phase, which may be referred to as the test phase, may have a duration of up to 1 minute, 2 minutes, 5 minutes, 10 minutes, 20 minutes, or more total time (including time engaged in an exploratory activity and time not engaged in an exploratory activity). Alternatively, the second phase may have a duration such that for up to 10 seconds, up to 20 seconds, up to 30 seconds, up to 40 seconds, up to 50 seconds, up to 60 seconds, up to 90 seconds, up to 180 seconds, or more total time the subject is engaged in an exploratory activity (e.g., probing, investigating and/or examining an object).

Often, in the first phase and/or second phase of the novel object recognition task the time spent investigating, probing and/or examining each object is quantified to provide a measure of the extent to which the subject engages in an exploratory activity with respect to an object (e.g., a novel object, a familiar object). For example, object recognition may be quantified as T_{Total}, T_{Novel}/T_{Total}, T_{Familiar}/T_{Total}, or T_{Novel}/T_{Total}−T_{Familiar}/T_{Total•}, wherein T_{Novel} is the time spent exploring a novel object, T_{Familiar} is the time spent exploring the familiar object and T_{Total} is the total time spent exploring objects. The parameter T_{Novel}/T_{Total} may be compared with the parameter T_{Familiar}/T_{Total•} to assess differences in exploratory activity directed at novel objects versus familiar objects.

### Novelty Odd Ball Test

In some embodiments, a “novelty oddball” task (e.g., a visual novelty oddball test) may be used as an exploratory activity. In some embodiments, this task is useful for studying cognitive processes such as novelty detection and selective attention. Generally, regulation of event-related brain potentials (ERPs) are observable in this task. In some embodiments, the novelty oddball test is used to assess neuronal activity during onset of an exploratory activity in primate subjects (e.g., human subjects). In a visual novelty oddball task, subjects are presented with different images, each image being presented for a relatively brief period of time. The images may be displayed in the perceptual environment through the use a computer monitor. In this task, the images typically include a simple “standard” image, a “novel” (highly salient) image, and a “dim” simple image. And, on a relatively small proportion of instances, a “target” image is presented, in response to which the subject responds by pressing a button.

EEG oscillations may be readily examined using this novelty oddball task. In some embodiments, the task may be used to assess whether images will evoke a certain EEG oscillation signatures at electrodes over the prefrontal cortex (or other brain region) in a subject. In some embodiments, the novelty oddball task allows for a comparison of signatures in EEG oscillations in humans with signatures in EEG oscillations in other subjects using other tasks. For example, a comparison may be made with signatures in EEG oscillations occurring in rodents during a novel object recognition task. In some embodiments, an informative comparison may be made between novel and dim image stimulus conditions, since these are matched for probability (both are infrequently presented) and task relevance (neither are targets).

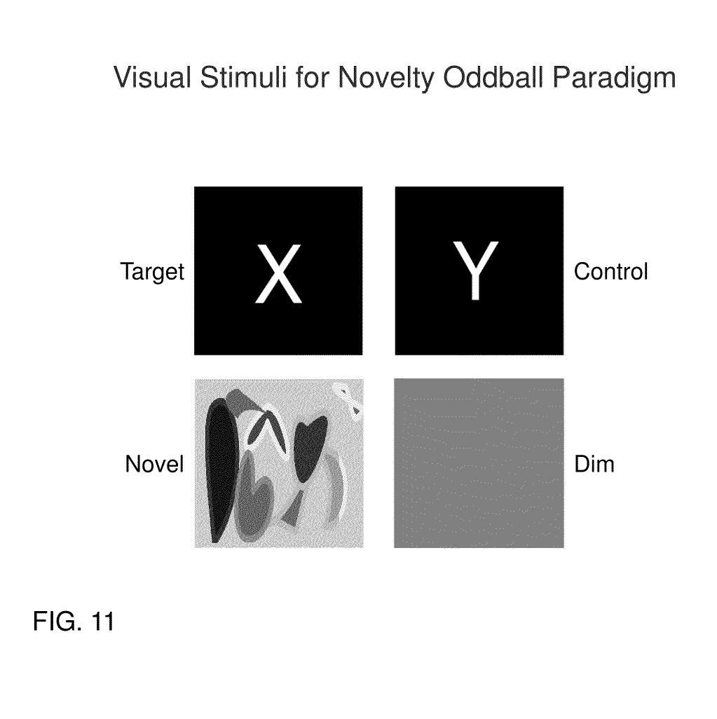

In some embodiments, the novelty oddball task involves subjects being seated in a comfortable chair in a darkened room. The stimuli may be presented on a computer monitor, situated a suitable distance (e.g., 100 cm) from the subject's nasion. In some embodiments, the image regime of Courchesne et al. (1975) may be used, in which 4 types of image stimuli are presented: targets (the letter “X”), standards (the letter “Y”), novels (complex, colored patterns), and “dims” (grey squares). Exemplary images are shown in FIG. 11. The task may be divided into blocks of trials. For example, each block of trials may include target images at a frequency 12%, novel images at a frequency of 12%, dim images at a frequency of 12%, and standard images at a frequency of 64%. The interval between each image presentation may be in a range of 1000 msec to 2500 msec. Each image may be presented for 200 msec to 1000 msec. In some embodiments, the subject's task is to make an affirmative indication each time a target stimulus is presented (e.g., by pressing a button, pulling a lever, toggling a switch, or tapping a screen when a target stimulus is presented).

During the novelty oddball task, EEG oscillations may be continuously recorded (e.g., at 512 Hz sampling rate) at standard electrode sites (e.g., using a scalp electrode set). In some embodiments, additional electrodes may be used for deriving the vertical and horizontal electro-oculograms (EOGs). Following data acquisition, the EEG oscillations may be processed by segmenting the oscillations into epochs encompassing stimulus onset (e.g., from −750 to 1298 msec relative to stimulus onset or other appropriate time segment). The epochs may be analyzed for artifacts, for example, by using a criterion of +/−90 μV for amplitude, or greater than 150 μV amplitude range or other appropriate criterion, on any channel. An independent component analysis or other suitable analysis may be applied to remove EOG and other artifacts (e.g., muscle artifacts, bad channels). Artifact-free epochs may be re-referenced to the average reference. ERPs may then be computed for each condition by averaging the single-trial epochs. Event-related time-frequency measures (e.g., evoked power, phase locking factor, and total power) may be computed, for example, by using the Morlet wavelet transform or other method known in the art and/or disclosed herein. In some embodiments, a predetermined range of frequencies may be analyzed at a particular frequency resolution. In some embodiments, frequencies in a range of 2-100 Hz may be analyzed at, for example, a 1 Hz resolution. Time-frequency maps of the evoked power, phase locking factor, and/or total power information may be produced, from which signatures of the EEG oscillations may be evaluated.

In some embodiments, differences in oscillatory activity between novel and dim images types may be evaluated to assess neuronal activity during an unconditioned response and evaluate signatures in EEG oscillations obtained during onset of an exploratory activity in the subject. For example, to determine whether oscillatory activity differs between the novel and dim conditions, a statistical mapping procedure (e.g., a non-parameter statistical mapping procedure) may be utilized to analyze time-frequency measures. T-tests may be computed at each time point for each frequency band between the novel and dim conditions, resulting in a time-frequency t-map (which may also be referred to as a time-frequency matrix). A permutation procedure may be employed to estimate probabilities of the values in t-maps. The permutation procedure may be used to obtain a time-frequency map of p-values for a novel vs. dim comparison. The time-frequency regions with significant p values (e.g., p values greater than 0.975 or less than 0.025, corresponding to a Type I error rate of 0.05) may be summed across channels to create a spatial histogram of novelty effects (novel>dim or novel<dim effects). Time-frequency clusters in the histogram may be thresholded (corresponding to a binomial probability of p<0.05) and visualized using topographic maps to detect signatures in EEG oscillations as manifest in the p-value clusters. Thus, in some embodiments, signatures in EEG oscillations may comprise information from statistical time-frequency maps.

### Methods for Determining the Effectiveness of a Therapeutic Agent

Methods for determining the effectiveness of a therapeutic agent for modulating neural activity in a subject are also provided. The methods typically involve administering a therapeutic agent (e.g., an approved drug, candidate therapeutic agent, etc.) to a subject identified as having, or being at risk of having, a cognitive disorder. The methods also typically involve inducing the subject to initiate an exploratory activity and recording electroencephalographic oscillations from the subject during onset of the exploratory activity. The presence or absence of a signature in the electroencephalographic oscillations is then evaluated. When absence of the signature is associated with the cognitive disorder, and treatment with the therapeutic agent (or candidate therapeutic agent) results in the presence of the signature, the therapeutic agent is identified as being effective for treating the cognitive disorder. In contrast, when presence of the signature is associated with the cognitive disorder, and treatment with the therapeutic agent (or candidate therapeutic agent) results in the absence of the signature, the therapeutic agent is identified as being effective for treating the cognitive disorder.

In some cases, the methods involve comparing the electroencephalographic oscillations that occur during onset of the exploratory activity to an appropriate standard in order to evaluate effectiveness of the therapeutic agent. Any appropriate standard may be used for evaluating the effectiveness of a therapeutic agent. For example, the appropriate standard may be one or more signatures in electroencephalographic oscillations that are observed in a subject who has not been treated with the therapeutic agent. In an alternative example, the appropriate standard may be one or more signatures in electroencephalographic oscillations that are observed in the subject prior to administration of the therapeutic agent.

Any of the methods disclosed herein for inducing a subject to engage in an exploratory activity may be used for determining the effectiveness of a therapeutic agent. Moreover, any of the methods disclosed herein for evaluating or identifying in a subject a signature in EEG oscillations (or the absence thereof) may be used for determining the effectiveness of a therapeutic agent. Typically, the methods are designed to evaluate a therapeutic agent's suitability for treating a cognitive disorder. Thus, the signature may serve as a biomarker (e.g., an electrophysiological endophenotype) for evaluating the effectiveness of an agent for treating a cognitive disorder.

As used herein the term, “disorder” refers to a disorder, disease, or condition. As used herein the term, “cognitive disorder” refers to a disorder, disease, or condition associated with one or more cognitive deficits. The term “cognitive deficit”, as used herein, refers to a deficiency in ability of a subject to engage in (or execute effectively) a task such as, for example, perception, memory, judgment, or reasoning. A cognitive deficit may be an impairment of attention, memory, learning, speed of learning or acquisition of data, flexibility, etc. In some embodiments, a subject may have one or more cognitive disorders.

Cognitive disorders may be caused by or associated with genetic factors, congenital factors, environmental factors (such as drug use, sleep deprivation, certain sensory inputs (e.g., excessive sound or excessive light), brain injuries, infection, etc.), or mental illness, among others. A cognitive disorder may be associated with a disease, such as, for example, schizophrenia, bipolar disorder, Alzheimer's disease, Parkinson's disease, Huntington's Disease, multiple sclerosis, Attention Deficit Hyperactivity Disorder (ADHD), autism, learning or memory disorder, brain injury, mental retardation or anxiety. In some cases, a cognitive disorder may be induced in a subject by treating the subject with a drug that impairs cognition (e.g., alcohol, apomorphine, d-amphetamine, methamphetamine phencyclidine (PCP), MK-801, ketamine, mescaline, lysergic acid diethylamide (LSD), psilocybin, scopolamine). For example, a subject may be treated with a drug or action (e.g., induced brain lesion or injury) that results in a cognitive disorder and the subject may then be tested using methods of the invention.

The subject may be a normal subject (e.g., wild-type subject) or a genetically altered subject (e.g., a knock-out subject, a knock-in subject, a transgenic subject) or a surgically altered subject, or a chemically altered subject, or a behaviorally altered subject (e.g., a sleep-deprived subject). The subject may be an inbred strain of a rodent having a particular phenotype. Typically, when a subject has a characteristic phenotype (e.g., a disease, a surgical induced brain damage, a disorder or condition) and/or known genotype (e.g., a mutation associated with disease), the subject is referred to as a “model” or “animal model” of the phenotype and/or known genotype. Thus, a subject exhibiting one or more symptoms of a cognitive disorder may be referred to herein as a “model of a cognitive disorder”. A subject exhibiting one or more symptoms of a cognitive deficit is referred to herein as a “model of a cognitive deficit”.

In some cases, a model of a disorder may be chemically induced. For example, a disorder may be a chemically induced neurological disorder. A disorder may be chemically induced with a drug that impairs glutamatergic function and mimics a psychotic state in the subject. Non-limiting examples of drugs that impair glutamatergic function include phencyclidine (PCP), MK-801, and ketamine. A disorder may be chemically induced with a drug that enhances dopaminergic function and mimics a psychotic state in the subject. Non-limiting examples of drugs that enhance dopaminergic function include apomorphine, D-amphetamine, and methamphetamine. A disorder may be chemically induced with a hallucinogenic drug that mimics positive symptoms associated with schizophrenia. Non-limiting examples of hallucinogenic drugs include mescaline, lysergic acid diethylamide (LSD), and psilocybin. A disorder may be chemically induced with a drug that impairs cholinergic function, which is believed to mimic aspects of the cognitive symptoms associated with schizophrenia. A non-limiting example of a drug that impairs cholinergic function is scopolamine.

Aspects of the methods involve comparing a signature in EEG oscillations in a subject with that of a control subject. As used herein, the term “control subject” refers to a subject having a known status, e.g., a known cognitive disorder status. An example of a control subject, though not intended to be limiting, is a subject that is a normal (e.g., non-cognitively impaired) subject. Thus, in some embodiments, an agent that, having been administered to a subject, results in the subject being more like that of a “normal” control subject in that a particular signature is present in EEG oscillations obtained from the subject, may be a candidate for treating a deficiency in a preparative neurological event in a subject.

Methods Identifying Test Agents that Modulate a Neural Activity and Improve Cognitive Function

Methods for identifying whether a test agent modulates neural activity and improves cognitive function in a subject are also provided. The methods typically involve administering a test agent to a subject, inducing the subject to initiate an exploratory activity and recording electroencephalographic oscillations from the subject during onset of an exploratory activity. Often the methods involve comparing the recorded electroencephalographic oscillations (or power or phase-locking information derived therefrom) to an appropriate standard, such that the results of the comparison identify or establish whether the test agent modulates the neural activity and improve cognitive function in the subject.

In some cases, the methods involve comparing signatures in electroencephalographic oscillations that occur during onset of an exploratory activity, to an appropriate standard in order to identify whether or not a test agent modulates neural activity in a manner that improves cognitive function. Any appropriate standard may be used for evaluating the effectiveness of a test agent. For example, the appropriate standard may be one or more signatures in electroencephalographic oscillations that are observed in a subject who has not been treated with the test agent. In an alternative example, the appropriate standard may be one or more signatures in electroencephalographic oscillations that are observed in the subject prior to administration of the test agent. Any of the methods disclosed herein for evaluating a subject during onset of an exploratory activity may be used for identifying test agents that have a desired activity.

As used herein, the term “test agent” refers to a compound or composition that is evaluated in an assay for its suitability as a candidate therapeutic agent. Without limitation, the following provides examples of test agents that may be used in the methods disclosed herein. Those of ordinary skill in the art will recognize that there are numerous additional types of suitable test agents that may be evaluated using the methods. Test agents can be small molecules (e.g., compounds that are members of a small molecule chemical library). The agents can be small organic or inorganic molecules of molecular weight below about 3,000 Daltons. The small molecules can be, e.g., from at least about 100 Da to about 3,000 Da (e.g., between about 100 to about 3,000 Da, about 100 to about 2,500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

Small molecules can be natural products, synthetic products, or members of a combinatorial chemistry library. A set of diverse molecules can be used to cover a variety of functions such as charge, aromaticity, hydrogen bonding, flexibility, size, length of side chain, hydrophobicity, and rigidity. Combinatorial techniques suitable for synthesizing small molecules are known in the art (e.g., as exemplified by Obrecht and Villalgrodo, Solid-Supported Combinatorial and Parallel Synthesis of Small-Molecular-Weight Compound Libraries, Pergamon-Elsevier Science Limited (1998)), and include those such as the “split and pool” or “parallel” synthesis techniques, solid-phase and solution-phase techniques, and encoding techniques (see, for example, Czarnik, A. W., Curr. Opin. Chem. Biol. (1997) 1:60). In addition, a number of small molecule libraries are publicly or commercially available (e.g., through Sigma-Aldrich, TimTec (Newark, Del.), Stanford School of Medicine High-Throughput Bioscience Center (HTBC), and ChemBridge Corporation (San Diego, Calif.).

In some embodiments, test agents are peptide or peptidomimetic molecules. In some embodiments, test agents include, but are not limited to, peptide analogs including peptides comprising non-naturally occurring amino acids, phosphorous analogs of amino acids, amino acids having non-peptide linkages, or other small organic molecules. In some embodiments, the test compounds are peptidomimetics (e.g., peptoid oligomers, e.g., peptoid amide or ester analogues, D-peptides, L-peptides, oligourea or oligocarbamate); peptides (e.g., tripeptides, tetrapeptides, pentapeptides, hexapeptides, heptapeptides, octapeptides, nonapeptides, decapeptides, or larger, e.g., 20-mers or more); cyclic peptides; other non-natural peptide-like structures; and inorganic molecules (e.g., heterocyclic ring molecules). Test agents can also be nucleic acids, including, e.g., shRNA, siRNA, microRNA, microRNA inhibitors (e.g., microRNA sponges), nucleic acid aptamers. In some embodiments, methods of the invention are used to evaluate, as test agents, “approved drugs”. An “approved drug” is any compound (which term includes biological molecules such as proteins and nucleic acids) which has been approved for use in humans by the FDA or a similar government agency in another country, for any purpose.

It will be understood that a therapeutic agent may reduce or eliminate a symptom of a disorder and may, but need not, eliminate the disorder. A therapeutic agent may delay onset of the disorder; shorten the duration of the disorder; eliminate the disorder in part; reduce the severity of one or more symptoms of the disorder; or eliminate the disorder entirely. Candidate therapeutic agents can be systematically altered, e.g., using rational design, to achieve (i) improved potency, (ii) decreased toxicity (improved therapeutic index); (iii) decreased side effects; (iv) modified onset of therapeutic action and/or duration of effect; and/or (v) modified pharmacokinetic parameters (absorption, distribution, metabolism and/or excretion).

The agents disclosed herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or as an aerosol. Thus, a variety of administration modes, or routes, are available. The particular mode selected will depend, of course, upon the particular test agent selected and the dosage required. Preferred modes of administration are parenteral and oral routes. The term “parenteral” includes subcutaneous, intravenous, intramuscular, intraperitoneal, and intrasternal injection, or infusion techniques. Other appropriate routes will be apparent to one of ordinary skill in the art.

According to the methods of the invention, the agents may be administered in a pharmaceutical composition. Administering the pharmaceutical composition of the present invention may be accomplished by any means known to the skilled artisan. In addition to the active agent, the pharmaceutical compositions of the present invention typically comprise a pharmaceutically-acceptable carrier. Pharmaceutically acceptable compositions can include diluents, fillers, salts, buffers, stabilizers, solubilizers and other materials which are well-known in the art. The term “pharmaceutically-acceptable carrier”, as used herein, means one or more compatible solid or liquid filler diluents or encapsulating substances which are suitable for administration to a human or lower subject. In preferred embodiments, a pharmaceutically-acceptable carrier is a non-toxic material that does not interfere with the effectiveness of the biological activity of the active ingredients. The term “compatible”, as used herein, means that the components of the pharmaceutical compositions are capable of being commingled with an agent, and with each other, in a manner such that there is no interaction which would substantially reduce the pharmaceutical efficacy of the pharmaceutical composition under ordinary use situations. Pharmaceutically-acceptable carriers must, of course, be of sufficiently high purity and sufficiently low toxicity to render them suitable for administration to the human or lower subject being treated.

Some examples of substances which can serve as pharmaceutically-acceptable carriers are sugars such as lactose, glucose and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; pyrogen-free water; isotonic saline; phosphate buffer solutions; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. The choice of pharmaceutically-acceptable carrier to be used in conjunction with the agents of the present invention is basically determined by the way the agent is to be administered. Pharmaceutically-acceptable carriers suitable for the preparation of unit dosage forms for oral administration and topical application are well-known in the art. Their selection will depend on secondary considerations like taste, cost, and/or shelf stability, which are not critical for the purposes of the subject invention, and can be made without difficulty by a person skilled in the art.

The agents of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

The pharmaceutically acceptable carrier employed in conjunction with the agents of the present invention is used at a concentration sufficient to provide a practical size to dosage relationship. The pharmaceutically-acceptable carriers, in total, may comprise from about 60% to about 99.99999% by weight of the pharmaceutical compositions of the present invention, e.g., from about 80% to about 99.99%, e.g., from about 90% to about 99.95%, from about 95% to about 99.9%, or from about 98% to about 99%.

### Diagnosis of Cognitive Disorders

Methods disclosed here may also be used for diagnosing, or aiding in diagnosing, a subject as having a cognitive disorder. For example, where a signature of EEG oscillation is indicative of normal cognitive function, absence of the signature (e.g., loss of one or more power maxima in a time-frequency map of power values) may indicate presence of a cognitive disorder. Similarly, where a signature of EEG oscillations is associated with a cognitive disorder, presence of the signature in EEG oscillations obtained from a test subject (e.g., a subject suspected of having the cognitive disorder) may indicated presence of the cognitive disorder in the subject.

Thus, in some embodiments, the diagnostic methods involve identifying a subject suspected of having a cognitive disorder or at risk of having the cognitive disorder and determining the presence or absence of a signature in electroencephalographic oscillations recorded from the subject during onset of an exploratory activity engaged in by the subject. In some embodiments, presence of the signature in the electroencephalographic oscillations is indicative of absence of a cognitive disorder in the subject. In some embodiments, absence of the signature in the electroencephalographic oscillations is indicative of presence of the cognitive disorder in the subject. In some embodiments, presence of the signature in the electroencephalographic oscillations is indicative of presence of a cognitive disorder in the subject. In some embodiments, absence of the signature in the electroencephalographic oscillations is indicative of absence of the cognitive disorder in the subject.

### Preparative Neurological Events as Biomarkers for Dopamine Receptor Activity

According to some aspects of the invention, it has been discovered that modulators of dopamine receptors affect a subject's neural activity during onset of an exploratory activity. Dopamine receptors are a class of metabotropic G protein-coupled receptors that are prominent in the CNS. The neurotransmitter dopamine is an endogenous ligand for dopamine receptors. Dopamine receptors are implicated in many neurological processes, including motivation, pleasure, cognition, memory, learning, and fine motor control, as well as modulation of neuroendocrine signaling. Genes encoding dopamine receptors include dopamine receptor D₁ gene (DRD1), dopamine receptor D₂ gene (DRD2), dopamine receptor D₃ gene (DRD3), dopamine receptor D₄ gene (DRD4), and dopamine receptor D₅ gene (DRD5). Alterations in one or more of these dopamine receptor genes, as well as abnormal dopamine receptor signaling and dopaminergic nerve function are implicated in several cognitive diseases. In some embodiments of the invention, signatures of electroencephalograph oscillations recorded during onset of an exploratory activity provide biomarkers for evaluating the effectiveness of test agents or therapeutic agents (e.g., approved drugs, candidate therapeutic agents) for modulating dopamine receptor activity and/or for treating cognitive diseases associated with altered dopamine receptor activity. Any of the methods provided herein for evaluating and identifying signatures of electroencephalograph oscillations recorded during onset of an exploratory activity may be used for identifying and/or characterizing agents that modulate dopamine receptor activity. Moreover any of the methods provided herein for evaluating and identifying signatures of electroencephalograph oscillations recorded during onset of an exploratory activity may be used for identifying and/or characterizing agents that modulate cognitive deficits associated with altered dopamine receptor activity.

According to some embodiments, methods are provided for identifying test agents that modulate dopamine signaling, and in particular, for identifying test agents that modulate dopamine signaling in GABAergic interneurons of the prefrontal cortex. In GABAergic interneurons of the PFC, D₄ receptor activation results in activation of calcineurin leading to suppression of interneuron activity via inhibition and internalization of α-amino-3-hydroxy-5-methyl-4-isoxazolepropionic acid receptors (AMPARs). In the PFC of subjects deficient in calcineurin (e.g., CNKO mice), it has been observed that mRNA coding for the D₄ receptor is over-expressed and that glutamatergic neurons are less active than in control (wild-type) subjects. In some embodiments, it is believed that D₄ receptor expression is up-regulated in this context to compensate for the reduced calcineurin activity. According to some embodiments, activation of D₄ receptors may restore the balance between excitation and inhibition within the PFC in the context of calcineurin deficiency and ameliorate the neurophysiological and cognitive deficits associated with this condition. Any of the methods provided herein for evaluating and identifying signatures of electroencephalograph oscillations recorded during onset of an exploratory activity may be used for identifying and/or characterizing agents that modulate dopamine signaling in GABAergic interneurons of the prefrontal cortex. Moreover any of the methods provided herein for signatures of electroencephalograph oscillations recorded during onset of an exploratory activity may be used for identifying and/or characterizing agents that modulate cognitive deficits associated with altered dopamine signaling in GABAergic interneurons of the prefrontal cortex.

### Systems and Components for Implementing Aspects of the Methods for Detecting and Evaluating Signatures in Electroencephalographic Oscillations

Aspects of the methods illustrated in FIG. 1 and FIG. 2, and disclosed elsewhere herein may be implemented in any of numerous ways. For example, the various methods or processes outlined herein may be coded as software that is executable on one or more processors that employ any one of a variety of operating systems or platforms. Such software may be written using any of a number of suitable programming languages and/or programming or scripting tools, and also may be compiled as executable machine language code or intermediate code that is executed on a framework or virtual machine. The MATLAB signaling processing toolbox (The MathWorks, Inc., Natick, Mass.) is an exemplary, but non-limiting, system that may be used for implementing certain aspects of the methods disclosed herein.

In this respect, aspects of the invention may be embodied as a computer readable medium (or multiple computer readable media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the invention discussed herein. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs, which when executed perform certain methods disclosed herein, need not reside on a single computer or processor, but may be distributed in a modular fashion among or between a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

As used herein, the terms “approximately” or “about” in reference to a number are generally taken to include numbers that fall within a range of 1%, 5%, 10%, 15%, or 20% in either direction (greater than or less than) of the number unless otherwise stated or otherwise evident from the context (except where such number would be less than 0% or exceed 100% of a possible value).

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element or a list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Words or phrases defined herein shall have the meanings herein ascribed unless an alternative meaning is clearly apparent from the language or context in which the word or phrase is used. Moreover, definitions provided for a word or phrase in one tense or form shall apply to other tenses or forms of the word or phrase.

Exemplary embodiments of the disclosure will be described in more detail by the following examples. These embodiments are exemplary of the disclosure, which one skilled in art will recognize is not limited to the exemplary embodiments.

### EXAMPLES

### Example 1

### Loss of CN Function is Associated with Deficits in Long-Term Object Recognition and High-Power Gamma Oscillations

Alterations in electroencephalographic oscillations (e.g., shifts in power) recorded from the prefrontal cortex (PFC) of mice could result from engagement of executive functions necessary for attending to and actively exploring a new environment; processes unrelated to executive function may also be involved. An integrated system was set up that combined automated real-time behavioral analysis (Topscan, CleverSys, Inc., Reston, Va.) together with multichannel electrophysiology equipment (MC_Rack, MultiChannel Systems, GmbH, Reutlingen, Germany) to analyze PFC activity during different phases of a novel object recognition (NOR) task in mice that were well habituated to the test apparatus.

EEGs were recorded from control and calcineurin heterozygous knock-out (CNₕₑₜKO) subjects during performance of a novel-object recognition task. Control and CNₕₑₜKO mice were exposed to two identical objects during a 10 min sample phase. After a 24 hour delay period, subjects were exposed to a familiar object and a novel object. During each phase, the behavior of each subject was monitored in real time and synchronized to recorded EEG oscillations (Topscan, CleverSys Inc.). As depicted in FIG. 4, an exploratory activity occurred when a subject's nose was orientated towards an object and was within a 2 cm radius surrounding the object for at least 200 msec, while the subject's center of mass (‘*’) was outside of 2 cm radius. The reason for excluding instances where a subject's center of mass was inside the radius, was to eliminate aberrant activity such as that in which a subject was sitting on an object and not actually exploring it.

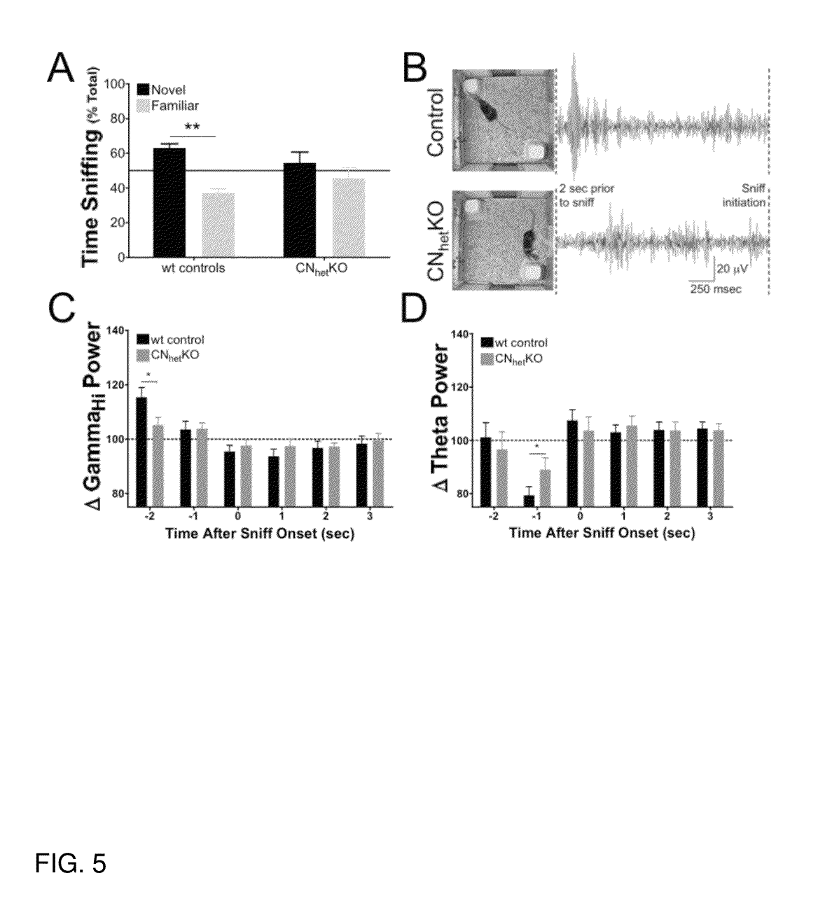

As depicted in FIG. 5A, it was observed that control subjects exhibited significantly more exploration of the novel object relative to the familiar object (t=3, df=9, p<0.01), whereas CNₕₑₜKO subjects did not exhibit significant difference in exploration between novel and familiar objects (t=1, df=12, p>0.05).

Differences between EEG oscillations in CNₕₑₜKO and normal control subjects were also evident in Gamma_{Hi} (65-90 Hz) bandpass filtered EEG traces (FIGS. 5B and 5C). An increase in amplitude and coherence was observed in the EEG oscillations recorded from the control subject during onset of the exploratory activity at approximately 2 seconds prior to initiation of the exploratory activity (an exploratory sniff). In contrast, the CNₕₑₜKO EEG exhibited no significant changes in EEG oscillation during the same time frame.

The area under the curve was computed from spectrograms to quantify power in different frequency bands [Theta (4-12 Hz) and Gamma_{HI} (65-90 Hz)] of the EEG oscillations. Gamma_{HI} in WT control subjects (FIG. 5C, black bars) was significantly increased relative to CNₕₑₜKOs (orange; F_{[5,22]}=8, p<0.0001) during the sample phase.

As depicted in FIG. 5D, wild-type control subjects exhibited a significantly greater drop in Theta power relative to CNₕₑₜKO (orange bars; F_{[5,22]}=6, p<0.0001) just prior to onset of the exploratory activity during the sample phase. These data indicated that loss of CN disrupts modulation of EEG spectral power in the PFC during the pre-initiation period of the exploratory behavior in mice.

These results indicate that exploratory activity-associated shifts in oscillatory power (e.g., Gamma_{HI}, Theta) in the PFC may reflect recruitment of the PFC in performance of executive functions related to attentional and/or decision making tasks that occur prior to initiation of an exploratory activity (e.g., during spontaneous object recognition). These data further indicate that measures of oscillation power in the PFC can be used as a determinant of cognitive function in the NOR task and in other cognitive tasks.

### Example 2

### Activation of D4 Receptors: Effects on PFC EEG and Behavior In Vivo

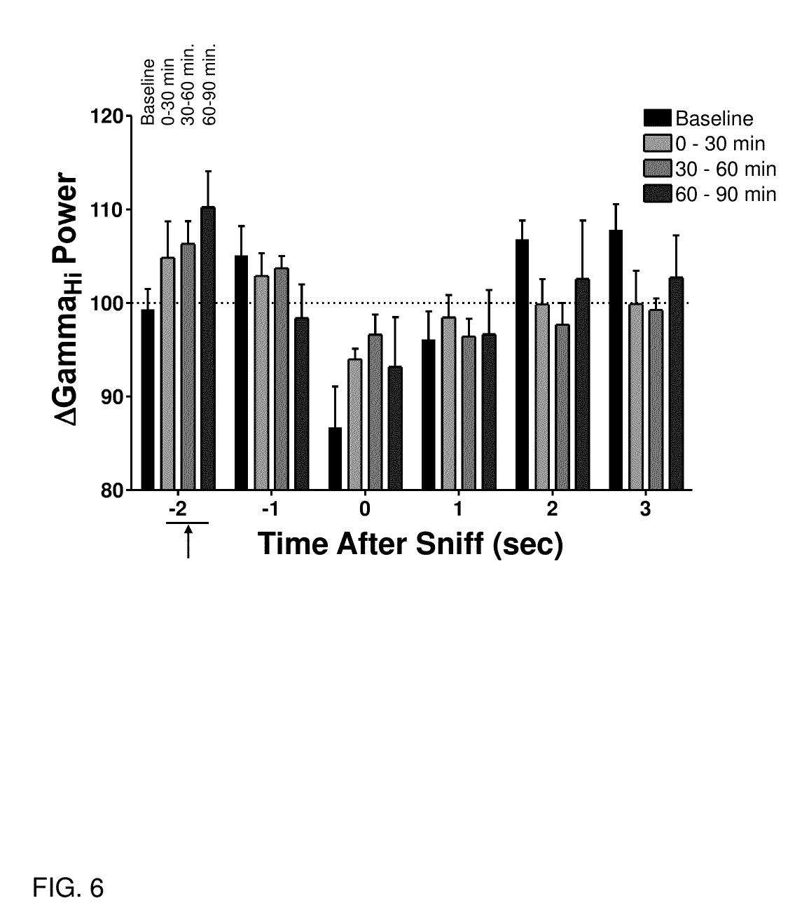

To determine whether activation of D4 receptors can have beneficial effects on changes in EEG power associated with exploratory behavior in the PFC, CNₕₑₜKO subjects were treated with PD 168077 (5 mg/kg) subcutaneously and EEG traces associated with exploratory activity (e.g., “sniffing”) in a novel object recognition task were analyzed. Exploratory activity in the presence of novel objects and familiar objects was analyzed for up to 90 min post-injection. As provided in Example 1 CNₕₑₜKO subjects fail to exhibit an increase in EEG power in the Gamma_{Hi} frequency band (65-90 Hz) prior to the exploratory activity, and this loss of Gamma power modulation is associated with a deficit in the novel object recognition cognitive paradigm. This result was obtained here again and is depicted in FIG. 6.

Injection of PD 168077 transiently restored normal Gamma responses in CNₕₑₜKO PFC elicited by object exploration (FIG. 6). Moreover, injection of PD 168077 (5 mg/kg, s.c.) 30 min prior to object exposure (familiarization phase) and again 30 min prior to novel/familiar object exposure (test phase after 24 hr) essentially completely restored performance of CNₕₑₜKO subjects in the novel object recognition cognitive paradigm (FIG. 6A, left panel).

These data indicate that a D4 receptor agonist can restore both synchronous network activity and cognitive function in CNₕₑₜKO subjects.

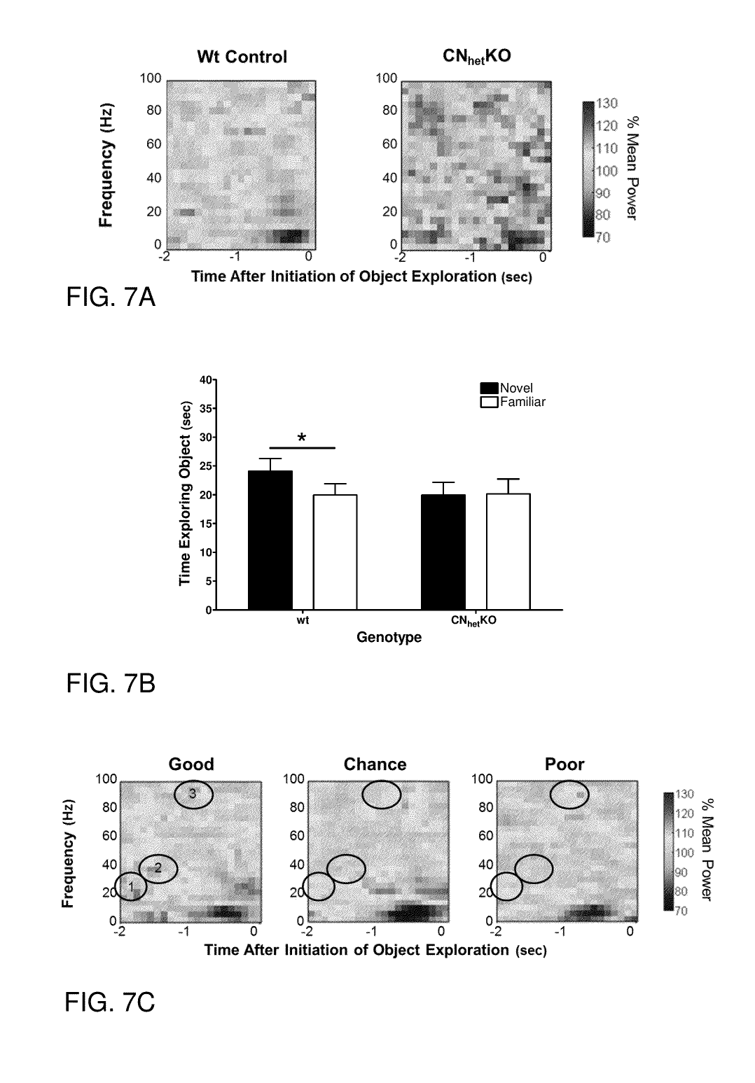

Time-frequency maps were produced of mean power for the normal and CNₕₑₜKO subjects. A specific signature was observed that indicated calcineurin disruption in object recognition. This signature was characterized by peaks in early beta (15-30 Hz) activity and late gamma_{Hi} (65-100) Hz activity. Alterations of the signature were thus observed in a genetic model of schizophrenia. Moreover, loss of calcineurin function resulted in diminished power in the ripple band frequencies (>100 Hz.) Taken together, these statistical analyses revealed significant clusters of neural activity in the wild-type PFC that are perturbed in the CNₕₑₜKO PFC. These results are depicted in the time-frequency maps of FIG. 7A. Differences in time-frequency maps of mean powers values between wild-type and CNₕₑₜKO mice correlated with differences in the relative time spent exploring novel versus familiar objects. Wild-type mice spent significantly more time exploring novel objects compared with familiar objects; whereas CNₕₑₜKO mice spent essentially the same amount of time exploring novel and familiar objects and this amount of time was comparable with the time that wild-type mice spent exploring the familiar objects. These results are depicted in FIG. 7B.

In a separate analysis, subjects were grouped based on observed performance in exploratory activity. Three groups of subjects were established: subjects exhibiting “good” performance, subjects exhibiting random (or “chance”) performance, and subjects exhibiting poor performance Good performance was defined as animals exhibiting greater than 20% preference to the novel object. Chance performance was defined as animals exhibiting between 20% preference for the novel object and 20% preference for the familiar object. Poor performance was defined as animals exhibiting a greater than 20% preference for the familiar object. It was observed that performance in the exploratory activity was associated with broad-band changes in neural activity as evident in time-frequency maps of mean EEG power (i.e., mean total power). A signature of EEG oscillations that was indicative of good cognitive performance was identified. This signature comprised early activation of (1) Beta (15-30 Hz) and Gamma_{Low} (30-55 Hz) bands, (2) reactivation of Gamma_{Low}, and late activation of Gamma_{Hi} (65-90 Hz) bands. These results are depicted in the time-frequency maps of FIG. 7B.

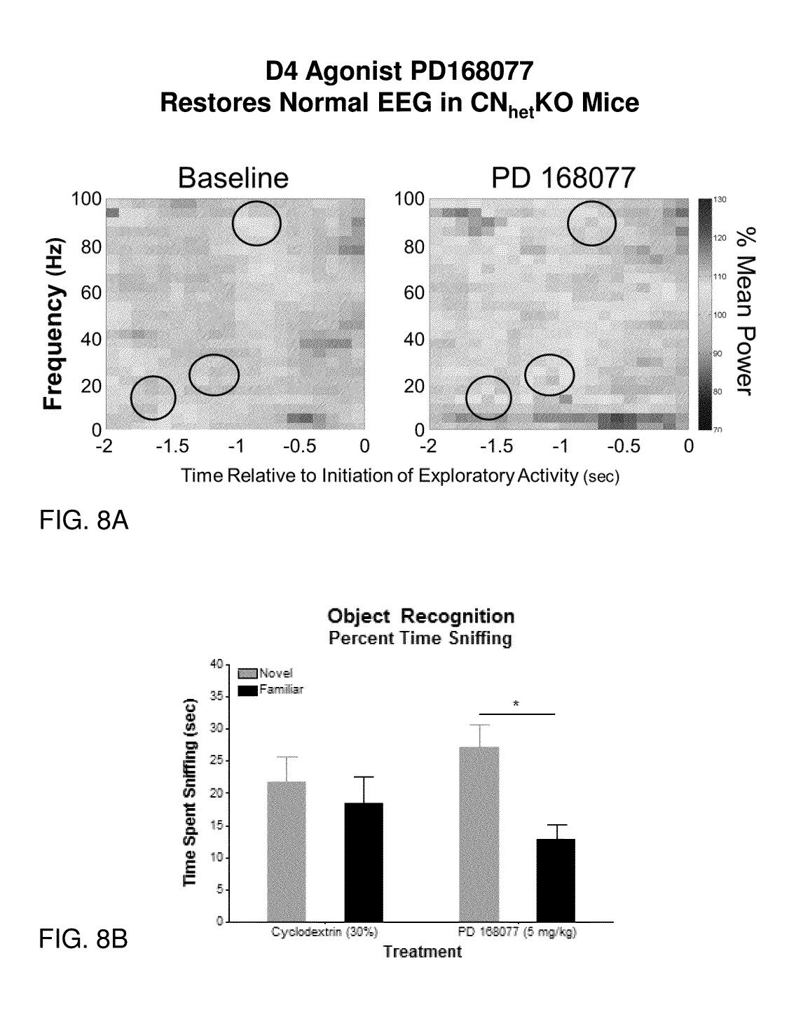

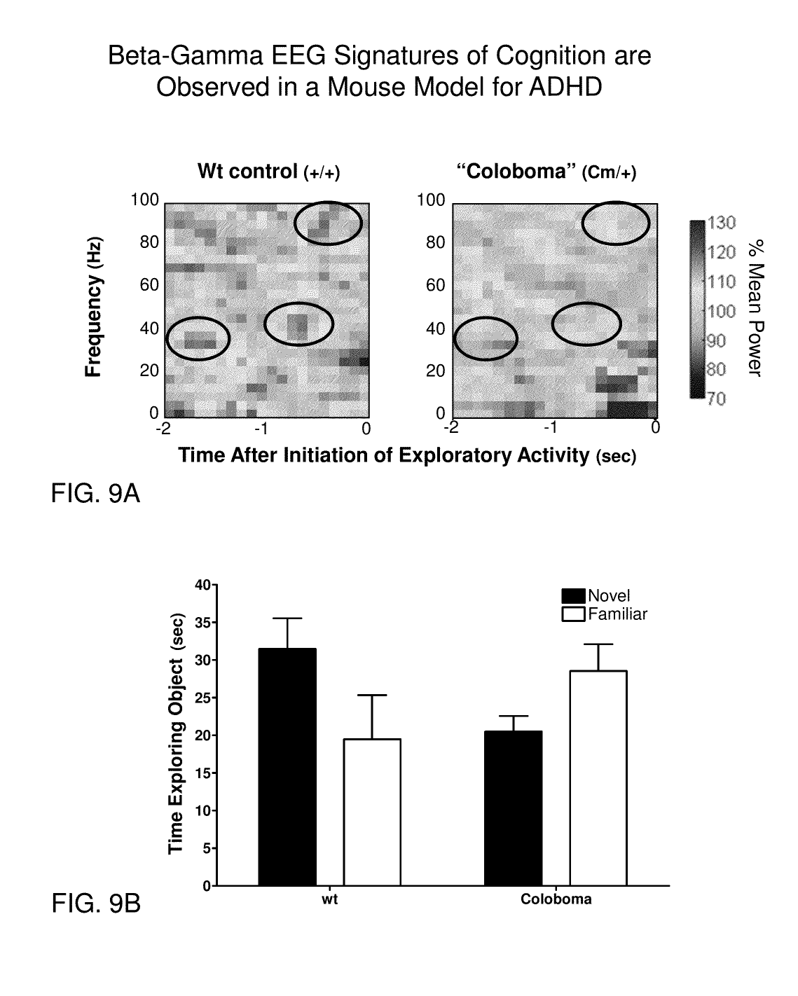

The effects of D4 agonist PD168077 on the EEG oscillations observed during onset of the exploratory activity in CNₕₑₜKO mice were evaluated. CNₕₑₜKO subjects were treated with PD 168077 (5 mg/kg) subcutaneously and EEG oscillations associated with exploratory activity in a novel object recognition task were analyzed. Exploratory activity in the presence of novel objects and familiar objects was analyzed for up to 90 min post-injection. Time-frequency maps of baseline conditions (no D4 agonist) and treatment conditions were evaluated. Surprisingly, the D4 agonist PD168077 restored the signature of EEG oscillation observed during onset of the exploratory activity, as shown in FIG. 7A. This result correlated with an overall improvement in the time spent exploring the novel object. CNₕₑₜKO mice treated with D4 agonist spent significantly more time exploring novel objects compared with familiar objects; whereas differences between time spent exploring novel objects versus familiar objects in CNₕₑₜKO mice treated with cyclodextrin control were not statistically significant. These results are depicted in FIG. 8B.

### Example 3

### Signatures in Electroencephalographic Oscillations During Onset of an Exploratory Activity in the Coloboma Mouse, a Genetic Model of ADHD

In mice, the mutation coloboma (Cm) corresponds to a contiguous gene defect that results in phenotypic abnormalities including spontaneous hyperactivity, head-bobbing, and ocular dysmorphology. The coloboma mouse has a contiguous deletion on chromosome 2 that is a syntenic with human chromosome 20p11-p12. This chromosomal region includes the following genes: Hao1; Pak 4, 7; Jag1; Fgfrl1; Txndc13; Rpl10, 21; Btbd3, 6; Snrpb2; Zfand1; Ankrd5; C20orf6, 7, 133; Hmgb1, 2; Rpl29; SNAP-25; Flrt3; Otor; Plc-1, 4; Mkks; Rpl26; and others.

Coloboma mice display impaired hippocampal synaptic plasticity, with perforant path long-term potentiation, and impaired transmitter release (e.g., impaired release of cortical glutamate, impaired release of DA and 5-HT in the dorsal striatum, and impaired Ach-induced CRF release in the hypothalamus). Coloboma mice exhibit a variety of cognitive phenotypes, including, for example, impaired latent inhibition and enhanced impulsivity. In addition, coloboma mice exhibit delays in achieving complex neonatal motor abilities and deficits in hippocampal physiology, which may contribute to learning deficiencies. The hyperkinesis is ameliorated by low doses of the psychostimulant D-amphetamine and can be rescued genetically by a transgene encoding SNAP-25. Together with syntaxin and synaptobrevin/VAMP, SNAP-25 constitutes a core protein complex integral to synaptic vesicle fusion and neurotransmitter release.

Time-frequency maps were produced of mean power for wild-type and Coloboma mice, which are a genetic model of ADHD. A specific signature was observed that indicated a disruption in object recognition associated with ADHD. This signature was characterized by peaks in early gamma_{Low} (30-40 Hz) activity and late gamma_{Hi} (80-100) Hz activity. The peaks were absent in the Coloboma mice. These analyses revealed significant clusters of neural activity in the wild-type PFC that are perturbed in the Coloboma PFC, and are depicted in the time-frequency maps of FIG. 7A. Differences in time-frequency maps of mean powers values between wild-type and Coloboma mice correlated with differences in the relative time spent exploring novel versus familiar objects. Wild-type mice spent significantly more time exploring novel objects compared with familiar objects; whereas Coloboma mice spent less time exploring novel than familiar objects (F[1,6]=8.4, p<0.05). These results are depicted in FIG. 7B.

### Example 4

### Signatures in Electroencephalographic Oscillations During Onset of an Exploratory Activity are Absent Following PCP Treatment

Phencyclidine (PCP) is a recreational dissociative drug. Formerly used as an anesthetic agent, PCP exhibits both hallucinogenic and neurotoxic effects. PCP is known for its primary action on ionotropic glutamate receptors, such as the NMDA receptor. As such, PCP is an NMDA receptor antagonist. NMDA receptors mediate excitation, however, studies have shown that PCP produces substantial cortical activation in humans and rodents.

PCP, like ketamine, also acts as a D2 receptor partial agonist. This activity may be associated with psychotic features of PCP intoxication, which is evidenced by the successful use of D2 receptor antagonists (such as haloperidol) in the treatment of PCP psychosis. PCP may also work as a dopamine reuptake inhibitor.

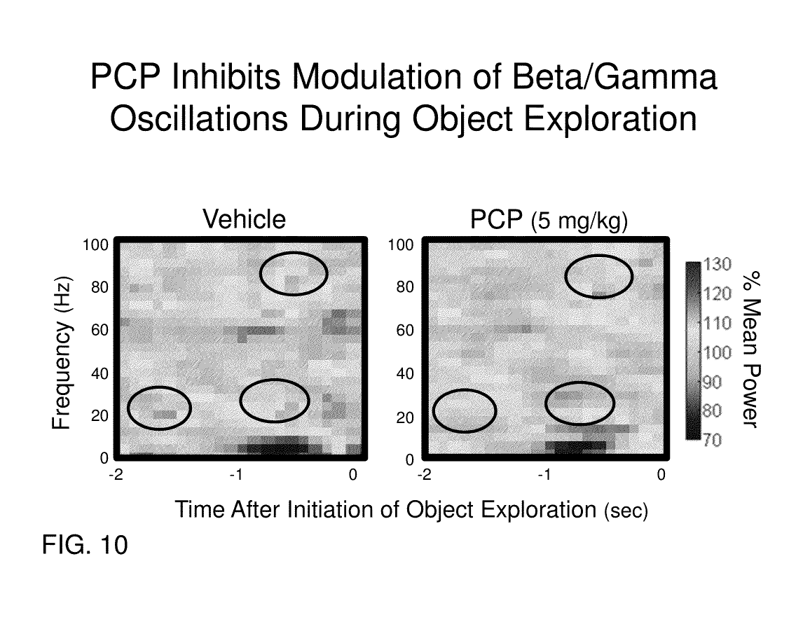

Time-frequency maps were produced of mean power for untreated wild-type mice and mice treated with PCP. A signature in EEG oscillations observed in untreated mice was absent following treatment with PCP. This signature was characterized by peaks in early beta (20-30 Hz) activity, gamma_{Low} (30-40 Hz) and gamma_{Hi} (80-100) Hz activity. These results are shown in the time-frequency maps of FIG. 10.

### Example 5

### Oscillatory Correlates of Novelty Detection in Humans

The presence or absence of a signature of novelty detection was determined in EEG oscillations from the prefrontal cortex (PFC) of humans. Shifts in EEG power associated with novelty detection were identified in EEG oscillations recorded from the PFC of humans. Ultimately, this novelty effect might is useful as a biomarker for, among other things, developing pharmaceutical treatments for schizophrenia and other mental health disorders.

A visual “novelty oddball” task (based on Courchesne et al. (1975)) was used as an exploratory activity in the human subjects. In this task subjects watched a computer monitor on which images were presented briefly. Most of the time, a simple “standard” image appeared. On a small proportion of trials another type of stimulus appeared: a simple “target” (to which the subject responds by pressing a button); a “novel”, highly salient image; or a “dim”, simple image. This task proved to be a fruitful approach for studying cognitive processes such as novelty detection and selective attention. The regulation of event-related brain potentials (ERPs) in this task are observable.

Oscillatory activity was examined in this novelty oddball task. Experiments were designed and conducted to assess whether images will evoke a gamma oscillation at electrodes over the PFC in human, similar to the results in mice was assessed. An informative comparison was made between the novel and dim stimulus conditions, since these are matched for probability (both are infrequent) and task relevance (neither are targets).

In this example, 12-15 healthy control subjects were subjected to the visual novelty oddball task, during which EEG oscillations were recorded. A Biosemi ActiveTwo EEG system was used. Data were analyzed using wavelet-based time-frequency analysis methods.

### Methods

### Subjects

Subjects were 12-15 healthy individuals recruited from a Schizophrenia Center's pool of control subjects. These individuals had participated in a number of EEG studies already. Subjects were selected without regard for ethnicity, and met the Schizophrenia Center's standard inclusion criteria: 1) age between 18-55 years; 2) right-handed (so that possible hemispheric lateralization effects would not be obscured by left-handers with reduced or reversed functional laterality); 3) no history of electroconvulsive treatment; 4) no history of neurological illness, including epilepsy; 5) no history of alcohol or drug dependence, nor abuse within the last year, nor long duration (>1 year) of past abuse (DSM-IV criteria); 6) no present medication for medical disorders that would have deleterious EEG, neurological, or cognitive functioning consequences; 7) verbal IQ above 75; 8) no alcohol use in the 24 hours prior to testing; and 9) English as a first language.

### Task

Subjects were seated in a comfortable chair in a darkened room. The stimuli was presented on a cathode ray tube computer monitor, situated 100 cm from the subject's nasion. Following Courchesne et al. (1975), there were 4 types of stimuli: targets (the letter “X”), standards (the letter “Y”), novels (complex, colored patterns), and “dims” (grey squares). Stimuli were measured approximately 3° X 3° of visual angle.

The task were divided into 6 blocks of 125 trials. Each block of trials consisted of 15 targets (12%), 15 novels (12%), 15 dims (12%), and 80 standards (64%). The interval between stimulus onsets was 1800 ms. Each stimulus was presented for 500 ms. The subjects' task was to press a button on the response box when a target stimulus is presented.

### EEG Recording and Processing

The EEG were continuously recorded at 512 Hz sampling rate using a 72-channel Biosemi ActiveTwo system at standard electrode sites. Additional electrodes were placed at below the left eye and at the outer canthi of the left and right eyes for deriving the vertical and horizontal electro-oculograms (EOGs), respectively.

Following data acquisition, the EEG were segmented into epochs from −750 to 1298 ms relative to stimulus onset. The epochs were analyzed for artifacts using a criterion of +/−90 μV for amplitude, or greater than 150 μV amplitude range, on any channel. Independent component analysis were applied to remove EOG and other artifacts (muscle artifacts, bad channels). The artifact-free epochs were re-referenced to the average reference. Following artifact correction/rejection, if a subject did not have at least 60 artifact-free trials in the target and novel conditions and 280 trials in the standard condition (i.e., 67% artifact-free trials in each condition), that subjects' data would not be further analyzed.

ERPs were computed for each condition by averaging the single-trial epochs. Event-related time-frequency measures (evoked power, phase locking factor, and total power) were computed using the Morlet wavelet transform. The range of frequencies to be analyzed were 2-100 Hz (1 Hz resolution).

### Statistical Analysis

A consideration in these studies was whether any oscillatory activity differed between the novel and dim stimulus types. To determine whether oscillatory activity differed between these conditions, a statistical non-parametric mapping procedure was utilized to analyze each of the 3 time-frequency measures. T-tests were computed at each time point for each frequency band between the novel and dim conditions, resulting in a time-frequency t-map.

A permutation procedure was employed to estimate the probabilities of the values in the t-map. This procedure has been shown to be an effective method for controlling for multiple comparisons (Maris & Oostenveld, 2007). The permutation procedure resulted in a time-frequency map of p-values for the novel vs. dim comparison. The time-frequency regions with significant p values (greater than 0.975 or less than 0.025, corresponding to a Type I error rate of 0.05) were summed across channels to create a spatial histogram of novelty effects (novel>dim or novel<dim effects). Time-frequency clusters in the histogram were thresholded at 8 channels (corresponding to a binomial probability of p<0.05) and 1 cycle duration at each frequency. The spatial distribution of the time-frequency clusters were visualized using topographic maps.

### Results

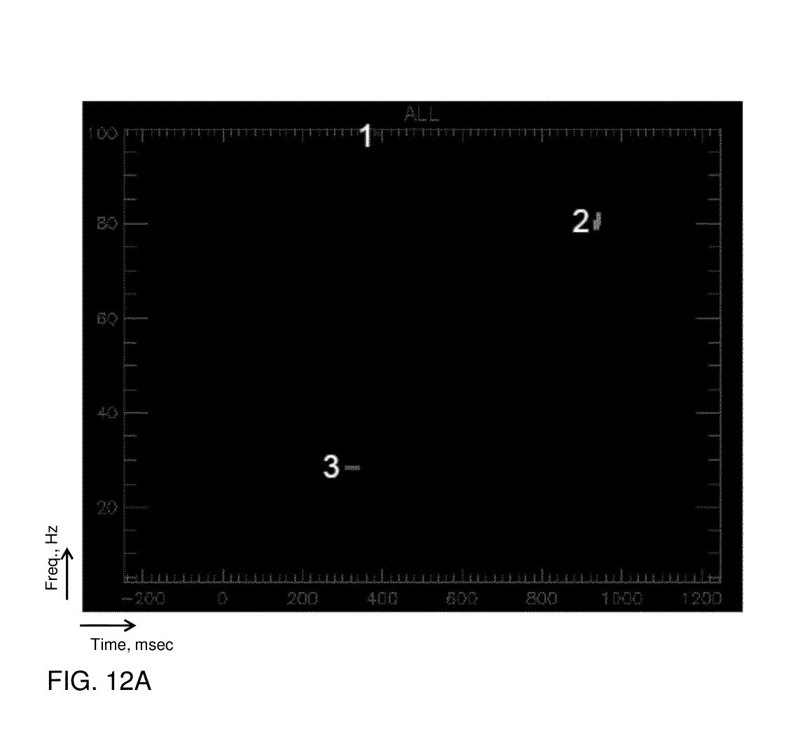

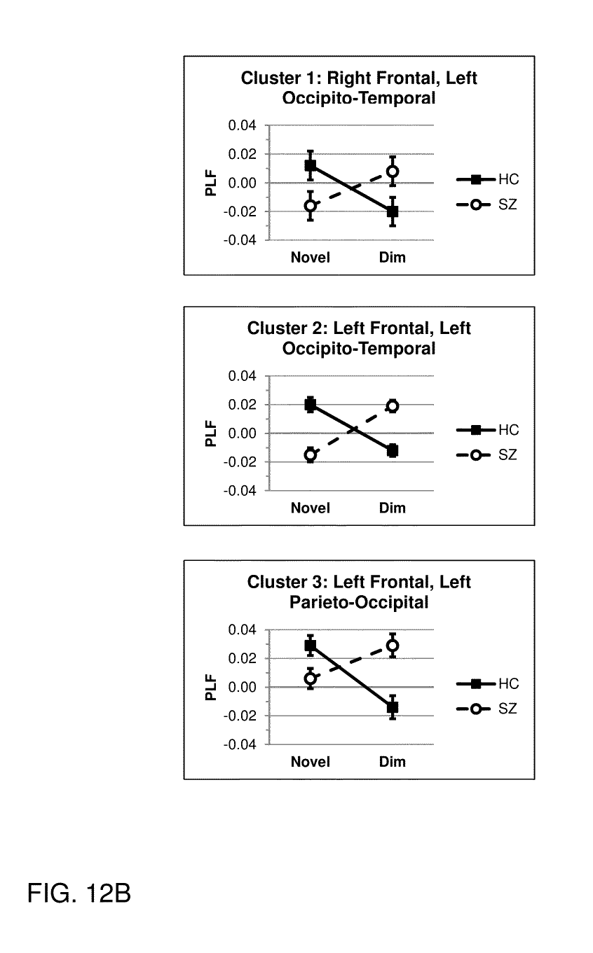

Three clusters of significant p-values were observed for comparisons of phase locking factors between novel images and dim images. A statistical time-frequency map was produced showing the three significant clusters. Cluster 1 comprised significant phase locking in the high gamma range (−99 Hz) at 384-392 msec following stimulus onset comparing novel images to dim images. Cluster 2 comprised significant phase locking in the high gamma range (79-82 Hz) at 929-949 msec following stimulus onset comparing novel images to dim images. The SZ showed decreased PLF in Clusters 1 and 2. Cluster 3 comprised significant phase locking in HC in the high beta range (28 Hz) at 306-343 msec following stimulus onset comparing novel images to dim images. Since Cluster 3 did not show a significant effect in SZ, it may be regarded as a relatively “pure” HC effect. These results are depicted in FIG. 12A. Statistical interactions were observed between healthy controls and schizophrenic subjects in each of the three clusters, as depicted in FIG. 12B, indicating that the signature in the statistical time-frequency map of p-values in healthy controls is altered (and thus absent) in schizophrenic subjects. There were interesting relationships (e.g., correlation patterns) observed in the clusters. For example, Cluster 3 was negatively correlated in healthy control with target response time (RT). So the Novel-Dim effect in this cluster decreased as RT increased (worse performance). This cluster was also negatively correlated with a neuropsych measure associated with working memory (Trails B, time spent).

It was observed that in healthy controls increased effects in clusters 1 and 3 (early high gamma and beta) correlated with better cognitive flexibility and task performance. In schizophrenic subjects increased effects in clusters 1 and 3 (early high gamma and beta) correlated with better task performance. It was also observed that increased effects in cluster 2 (late high gamma) correlated with worse cognitive flexibility and task performance. Cluster 2 negatively correlated, in health controls, with Clusters 1 and 3.

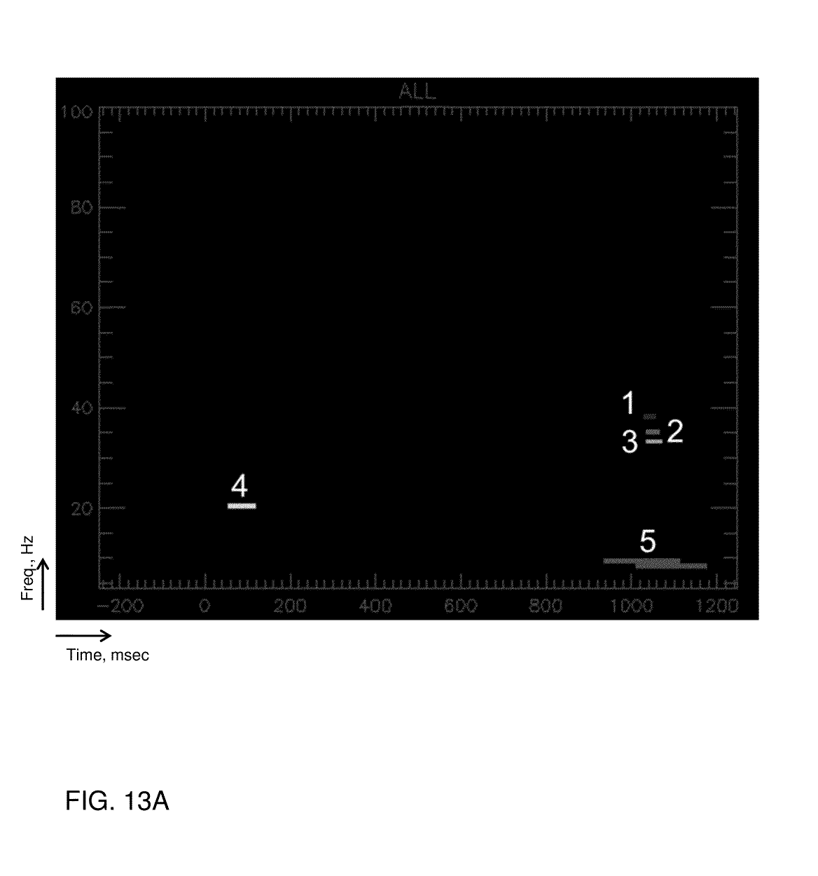

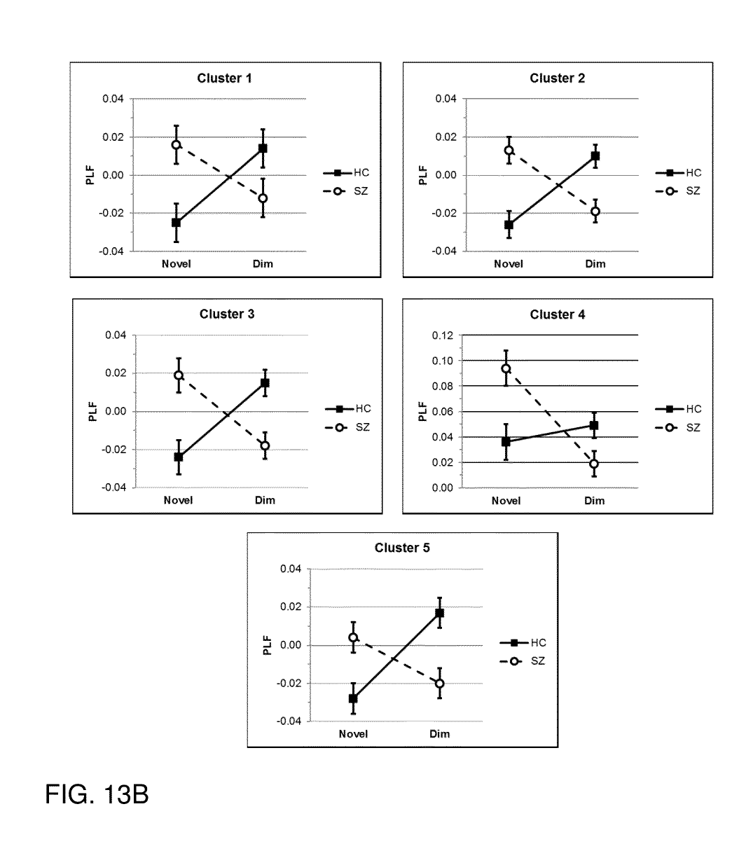

As depicted in FIGS. 13A and 13B, five clusters of significant p-values were observed for the Group X Stimulus interaction in which Novel minus Dim phase locking factor values were higher for SZ than HC. The earliest cluster occurred in the beta range (Cluster 4: 20 Hz, 53-119 ms) at mainly frontal electrodes, and represented a Novel>Dim effect for SZ with no effect for HC. The remaining 4 clusters occurred late in the epoch (936-1176 ms) in the alpha (Cluster 5: 8-9 Hz) and gamma (Clusters 1-3: 33-38 Hz) bands. The alpha cluster was present at distinct groups of fronto-central and occipito-temporal electrodes, while the gamma clusters were distributed across occipital, parietal, central, and frontal electrodes. The alpha and gamma clusters all showed the same pattern of effects (Novel>Dim for SZ and Dim>Novel for HC). Given the temporal coincidence and similarity of effects of the alpha and gamma clusters, these clusters might represent a cross-frequency interaction between the alpha and gamma bands.

In temporal order, the following clusters are depicted in FIG. 13A-B:

Cluster 4: Early beta (53-119 ms, 20 Hz). SZ: Nov>Dim
Cluster 5: Late alpha (936-1176 ms, 8-9 Hz). HC: Dim>Nov. SZ: Nov>Dim.
Cluster 1: Late low gamma (1029-1055 ms, 38 Hz). HC: Dim>Nov. SZ: Nov>Dim.
Cluster 2: Late low gamma (1033-1065 ms, 35 Hz). HC: Dim>Nov. SZ: Nov>Dim.
Cluster 3: Late low gamma (1035-1070 ms, 33 Hz). HC: Dim>Nov. SZ: Nov>Dim.

### REFERENCES

Courchesne E, Hillyard S A, Galambos R (1975). Stimulus novelty, task relevance, and the visual evoked potential in man. Electroencephalogr Clin Neurophysiol 39:131-143.
Demiralp T, Ademoglu A, Comerchero M, Polich, J (2001). Wavelet analysis of P3a and P3b. Brain Topogr 13:251-267.
Maris E, Oostenveld R (2007). Non-parametric statistical testing of EEG- and MEG-data. J Neurosci Meth 164:177-190.

### Example 6

### Comparison of Mouse and Human Signatures Based on Time-Frequency Maps

An assessment was made of similarities between human and mouse studies examining differences in signatures in EEG oscillation during onset of exploratory activity. Mouse experiments were conducted using the novel object recognition task, in which comparisons were made between normal (wild-type) mice and diseased (the CNₕₑₜKO, schizophrenic model). Human experiments were conducted using the novelty odd-ball test, in which comparisons were made between normal humans and diseased (schizophrenic) humans. The proportion of subjects showing peaks (i.e., maxima) at (10-30 Hz—peak 1) and (50-90 Hz—peak 2) was determined for both mice and human subjects. For mice peaks were in total power; whereas for humans peaks were clusters of significant p-values in comparisons between novel and dim images. The average time between peaks 1 and 2 was determined. The average time of peak 1 relative to onset of exploratory activity was compared. The average time of peak 2 relative to onset of exploratory activity was compared. In both mice and human most subjects exhibited peaks 1 and 2, and there was a significant decrease in the presence of peaks 1 and 2 in schizophrenic subjects. These results are outlined in Table 3 below.

TABLE 3
Comparison of mouse and human signatures of EEG oscillations

| | Mouse | | Human | |
|---|---|---|---|---|
| | Normal | Diseased | Normal | Diseased |
| Proportion of subjects | 88% | 67% | Peak 1: 100% | Peak 1: 64% |
| showing Peak 1 (10-30 Hz) | | | Peak 2: 93% | Peak 2: 86% |
| and Peak 2 (50-90 Hz) | | | | |
| Average Time Between Peak | 693 ± 74 | 615 ± 143 | 78 | 78 |
| 1 and Peak 2 (msec) | | | | |
| Average Time of Peak 1 | −1,528 ± 77 | −1,397 ± 141 | 306 | 306 |
| Relative to Onset of | | | | |
| Exploratory Activity (msec) | | | | |
| Average Time of Peak 2 | −834 ± 90 | −781 ± 136 | 384 | 384 |
| Relative to Onset of | | | | |
| Exploratory Activity (msec) | | | | |

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

1. A method comprising:
determining the presence or absence of a signature in electroencephalographic oscillations recorded from a subject during onset of an exploratory activity engaged in by the subject, wherein presence of the signature in the electroencephalographic oscillations is indicative of absence of a cognitive disorder in the subject, and wherein absence of the signature in the electroencephalographic oscillations is indicative of presence of the cognitive disorder in the subject.

2. A method comprising:
administering a test agent to subject who is identified as having a cognitive disorder; and
determining the presence or absence of a signature in electroencephalographic oscillations recorded from the subject during onset of an exploratory activity engaged in by the subject after having been administered the test agent,
wherein presence of the signature in the electroencephalographic oscillations is indicative of effectiveness of the test agent in treating the cognitive disorder, and wherein absence of the signature in the electroencephalographic oscillations is indicative of a lack of effectiveness of the test agent in treating the cognitive disorder.

3. A method of diagnosing, or aiding in diagnosing, a subject as having a cognitive disorder comprising:
identifying a subject suspected of having a cognitive disorder or at risk of having the cognitive disorder; and
determining the presence or absence of a signature in electroencephalographic oscillations recorded from the subject during onset of an exploratory activity engaged in by the subject,
wherein presence of the signature in the electroencephalographic oscillations is indicative of absence of a cognitive disorder in the subject, and wherein absence of the signature in the electroencephalographic oscillations is indicative of presence of the cognitive disorder in the subject.

4. The method of claim 1 further comprising:
recording electroencephalographic oscillations from the subject during onset of the exploratory activity.

5. The method of claim 1 further comprising:
stimulating the subject to engage in the exploratory activity.

6. The method of claim 1, wherein the signature is based on power of the electroencephalographic oscillations or a phase-locking characteristic of the electroencephalographic oscillations.

7. The method of claim 1, wherein the signature is a first maxima of power of the electroencephalographic oscillations occurring within a first frequency band followed by a second maxima of power of the electroencephalographic oscillations occurring within a second frequency band.

8. The method of claim 7, wherein the second maxima occurs 10 milliseconds to 1000 milliseconds following the first maxima.

9. The method of claim 7, wherein the first frequency band comprises lower frequencies than the second frequency band.

10. The method of claim 7, wherein the first frequency band is in a range of 10 Hz to 30 Hz.

11. The method of claim 7, wherein the second frequency band is in a range of 60 Hz to 100 Hz.

12. The method of claim 1, wherein the presence or absence of the signature is determined in electroencephalographic oscillations recorded from 3 seconds prior to initiation of the exploratory activity to 3 seconds after initiation of the exploratory activity.

13. The method of claim 1, wherein the exploratory activity is engaged in by the subject when an appropriate stimulus is in the perceptual environment of the subject.

14. The method of claim 13 further comprising setting the appropriate stimulus in the perceptual environment of the subject.

15. The method of claim 13, wherein the appropriate stimulus is an object or image.

16. The method of claim 13, wherein the appropriate stimulus comprises a light, sound, odorant, tastant, or tactile stimulant and/or induces the subject's sense of sight, hearing, smell, taste or touch.

17. (canceled)

18. The method of claim 13, wherein, prior to the appropriate stimulus being set in the perceptual environment, the subject has not been exposed to the appropriate stimulus for at least 12 hours, at least 24 hours or at least 48 hours.

19. The method of claim 13, wherein, prior to the appropriate stimulus being set in the perceptual environment, the subject has not been exposed to the appropriate stimulus.

20. The method of claim 19, wherein the exploratory activity involves a body portion of the subject being maintained within a first distance from the object for a first period.

21.-33. (canceled)

34. The method of claim 1, wherein the cognitive disorder is schizophrenia, bipolar disorder, Alzheimer's disease, Parkinson's disease, Huntington's disease, multiple sclerosis, Attention Deficit Hyperactivity Disorder (ADHD), autism, a learning disorder, a memory disorder, an injury, or anxiety.

35.-52. (canceled)
